(19) United States
(12) Patent Application Publication
(10) Pub. No.: US 2025/0137052 A1
(43) Pub. Date: May 1, 2025

(54) ATF3 AS A BIOMARKER OF CENTRAL NERVOUS SYSTEM INJURY

(71) Applicant: The Regents of the University of California, Oakland, CA (US)

(72) Inventors: Jonathan Z. Pan, Oakland, CA (US); Zhonghui Guan, Oakland, CA (US); Hua Su, Oakland, CA (US)

(21) Appl. No.: 18/693,883

(22) PCT Filed: Sep. 21, 2022
(86) PCT No.: PCT/US2022/076806

§ 371 Date: Mar. 20, 2024

Related U.S. Application Data
(60) Provisional application No. 63/246,668, filed on Sep. 21, 2021.

(51) Int. Cl. C12Q 1/6883; G01N 33/68

(52) U.S. Cl. CPC C12Q 1/6883; G01N 33/6896; C12Q 2600/112; C12Q 2600/158; G01N 2333/4703; G01N 2800/2871; G01N 2800/56

(57) ABSTRACT

The current medical managements of CNS injuries, such as spinal cord injury (SCI), IO traumatic brain injury (TBI) or stroke are far from ideal. Clinical trials in acute CNS injuries are challenging because of multiple barriers, such as heterogeneity of the injury and limitations of the imaging test and functional examination to identify the degrees of injuries. Therefore, there are urgent needs for identifying a biomarke that can be employed by clinicians to objectively stratify patients' initial severity of injuries as well as monitor injury progression and response to treatment.

### CROSS-REFERENCE TO RELATED APPLICATIONS

This application claims priority to U.S. Provisional Application No. 63/246,668, filed Sep. 21, 2021, which is incorporated by reference in its entirety.

### BACKGROUND

The current medical managements of CNS injuries, such as spinal cord injury (SCI), traumatic brain injury (TBI) or stroke are far from ideal. Clinical trials in acute CNS injuries are challenging because of multiple barriers, such as heterogeneity of the injury and limitations of the imaging test and functional examination to identify the degrees of injuries. Therefore, there are urgent needs for identifying a biomarker that can be employed by clinicians to objectively stratify patients' initial severity of injuries as well as monitor injury progression and response to treatment. Biomarker utilization in clinical medicine has been successful in diagnosis of tissue damage (cardiac enzymes such as troponin), tumor classification and severity (CEA, carcinoembryonic antigen; PSA, prostate-specific antigen) and atherosclerotic cardiovascular disease risk and response to therapy (blood lipid profile after cholesterol-lowering medication) (1-4).

Extensive previous work has identified several biomarkers for patients with acute CNS injuries (5-9). In SCI, serum and cerebrospinal fluid (CSF) glial fibrillary acidic protein (GFAP), neurofilament protein (NF), and peripheral cytokine and chemokine levels (10, 11), as well as microRNA or RNA profiles in circulating leukocytes (7) have been investigated as the biomarkers. In TBI, blood levels of GFAP and ubiquitin C-terminal hydrolase L1 (UCH-L1) have been recently FDA (federal drug administration)-approved as the biomarker for intracranial lesion (bleeding) after mild TBI and concussion (12), although the combined biomarker measurement only helps to identify the bleeding from head injury, instead of diagnosis of concussion or TBI. Similarly, GFAP, S100β, and neuron-specific enolase (NSE), among others, have been investigated as the biomarkers for stroke (9). Of note, GFAP and S100β are expressed in astrocyte, and NF, UCH-L1, and NSE are expressed in all neurons, no matter whether the neurons are injured or not. Thus, none of the biomarkers currently investigated in CNS injuries are expressed specifically in the injured neurons.

### BRIEF SUMMARY

This section provides a summary of certain aspects of the disclosure. The invention is not limited to embodiments summarized in this section.

The disclosure is based, at least in part, on the findings that ATF3 is induced only in the injured neurons shortly after CNS injuries, and can be measured in human blood and CSF, and is elevated in patients with spinal cord injury (CSI), hemorrhagic or ischemic stroke, or cardiac arrest, but not control patients. Therefore, ATF3 serves as a biomarker for evaluating the degree of neuronal injury at the acute stage of CNS injuries and can serve as a biomarker fro clinical CNS injuries in general. We additionally identified that ATF3 has a neuroprotective function after SCI or stroke in mouse models.

In one aspect, the disclosure provides a method of assessing severity of a spinal cord injury (CSI), the method comprising determining the level of ATF3 polypeptide in a cerebrospinal fluid (CSF) or blood sample, e.g., serum sample, from a subject that has a CSI injury; comparing the level of ATF3 polypeptide to a reference scale determined from a population of patients having American Spinal Injury Association Impairment Scale (AIS) Grades ranging from Grade A to Grade E; and classifying the severity of CSI injury, wherein: the subject is classified as having severe injury if the level of ATF3 polypeptide exceeds a threshold value for Grade A injury; the subject is classified as having moderate injury if the level of ATF3 polypeptide exceeds a threshold value for Grade C injury, but is less than the threshold value for Grade A injury; or the subject is classified as having mild injury if the level of ATF3 polypeptide is below the threshold value for Grade C injury. In some embodiments, the samples is a CSF sample. In some embodiments, the sample is a blood sample, e.g., a serum sample.

In a further aspect, the disclosure provides a method of assessing severity of a central nervous system injury, the method comprising determining the level of ATF3 polypeptide or RNA in a cerebrospinal fluid (CSF) or blood sample, e.g., serum, from a subject that has a CNS injury; comparing the level of ATF3 polypeptide or RNA to a reference scale derived from a population of patients having mild to severe CNS injury; and classifying the severity of injury, wherein: the subject is classified as having severe injury if the level of ATF3 polypeptide or RNA is in the top tertile of the reference scale; the subject is classified as having moderate injury if the level of ATF3 polypeptide or RNA is in the middle tertile of the reference scale; or the subject classified as having mild injury if the level of ATF3 polypeptide or RNA is in the lowest tertile of the reference scale. In some embodiments, the CNS injury is a spinal cord injury (CSI). In some embodiments, the sample is a blood sample, e.g., a serum sample. In some embodiments, the sample is a CSF sample. In some embodiments, the method comprises determining the level of ATF3 polypeptide. In some embodiments, the CNS injury is stroke, e.g., ischemic stroke. In some embodiments, the CNS injury is hemorrhagic stroke. In some embodiments, the subject had had cardiac arrest.

In another aspect, the disclosure provides a method of detecting ATF3 in a CSF sample or blood sample, e.g., serum sample, from a subject having a CNS injury, the method comprising contacting the CSF sample or blood sample, e.g., serum sample, with an agent that specifically binds to ATF3; and detecting the agent bound to ATF3. In some embodiments, the agents is an antibody. In some embodiments, the agents is an aptamer. In some embodiments, the sample is a CSF sample. In some embodiments, the subject has a CSI. In some embodiments, the subject has had a stroke. In some embodiments, the stroke is an ischemic stroke. In some embodiments, the stroke is hemorrhagic stroke. In some embodiments, the patient has had cardiac arrest.

Other objects, features, and advantages of the present invention will be apparent to one of skill in the art from the following detailed description and figures.

### BRIEF DESCRIPTION OF THE DRAWINGS

FIG. 1A-F. FIG. 1. ATF3 induction after spinal cord injury (SCI) and ischemic stroke. (A) Heat map of spinal cord RNA sequencing results before (sham) and 4 hours after SCI showing 177 differentially expressed genes (DEG) with more than 1.5-fold changes (160 upregulated and 17 downregulated). (B) Top 20 in GO analysis of DEG, among which Atf3 is involved multiple major pathways including MAPK cascade, positive regulation of cell death, and regulation of extrinsic apoptotic signaling pathway, negative regulation of phosphorus metabolic pathway. (C) Volcano plots of spinal cord RNA-Seq results showing that Atf3 is one of the most significantly upregulated genes 4 hours after SCI (adjusted BHFDR p<0.05). (D) qRT-PCR confirms the increased Atf3 expression 4 h after SCI. (E) Western blot and the quantification showing that ATF3 protein was induced in cortex 3 days after ischemic stroke. Data are presented as mean±S.E.M., unpaired two-tailed t-test, **p<0.01, and ***p<0.001, n=3. CT: control.

FIG. 2A-E ATF3 is mainly induced in neurons after CNS injuries. (A) Representative immunohistochemical staining of neuronal marker NeuN and ATF3 in rat injured hemi-cord 1 day post injury. ATF3 expression is mainly detected in the NeuN⁺ neurons at ventral horn gray matter in injured hemi-cord. Scale bar=200 μm. (B) Magnification of the squared areas in (A) showing that ATF3 co-localizes with NeuN (ATF⁺/NeuN⁺ positive cells marked by arrows). Scale bar=40 μm. (C) Representative immunohistochemical staining of NeuN and ATF3 in the peri-infarct ischemia region (FIG. 10) 1-3 days after pMCAO stroke in mice. ATF3 expression is mainly detected in the NeuN⁺ neurons. Scale bar=50 μm. (D) Quantification of the percentage of ATF3⁺/NeuN⁺ double positive cells among total ATF3⁺ cells. At 1 day after both SCI and stroke, 100% ATF3⁺ cells are NeuN⁺ neurons. 2 days to 3 days post-stroke, the percentage of ATF3⁺ cells that were neurons were 95% and 90%, respectively. Data are presented as mean±S.E.M., one-way ANOVA with Tukey's test, ***p<0.001, n=6 in each group. (E) Co-immunostaining of ATF3 and Fluoro Jade C (FJC, a known marker for degenerating neurons) in the peri-infarct ischemia region 1-3 days after pMCAO stroke in mice shows that all FJC⁺ cells are ATF3⁺ (arrowheads) with enlarged and irregularly shaped nuclei. However, many ATF3⁺ cells are FJC⁻ (arrows), and these cells have small nuclei with regular shapes. Scale bar=50 μm.

FIG. 3A-E. ATF3 expression in few non-neuronal cells after SCI or stroke. (A) Representative immunohistochemical (IHC) staining of ATF3 and CD68 in injured hemi-cord 1 day post injury (dpi). There is no ATF3 signals in CD68⁺ cells (microglia). Scale bar=40 μm. (B) Representative IHC staining of ATF3 and CD68 in the peri-infarct ischemia region (FIG. 10) 1-3 days after pMCAO in mice. Scale bar=50 μm. (C) Quantification of ATF3 and CD68 double positive cells. 1 day after both SCI or stroke, none of ATF3⁺ cells was CD68 positive. There were 0.1% and 0.25% ATF and CD68 double positive cells at 2 days and 3 days post-stroke, respectively. Data are presented as mean±S.E.M., one-way ANOVA with Tukey's test, *p<0.05, **p<0.01, n=6. (D) Representative IHC staining of ATF3 and GFAP in injured hemi-cord 1 dpi. There is no ATF3 signals in GFAP⁺ astrocytes. Scale bar=40 μm. (E) Representative IHC staining of ATF3 and GFAP in peri-infarct ischemia region 1 to 3 days after ischemic stroke. Scale bar=50 μm.

FIG. 4A-E Increased ATF3 protein levels in cerebrospinal fluid and plasma after SCI or stroke. ELISA results showing ATF3 protein was detectable in blood (A), CSF (B) and spinal cord (C) in control mice, and its level was increased significantly 1 day post SCI. ATF3 protein level was significantly elevated in the blood 6 hours, 1 day and 3 days after pMCAO (D), as well as in CSF 3 days after pMCAO (E). (F) ATF3 levels are elevated and correlated with injury severity (CT: sham, mild: 75 Kdyne, severe: 150 Kdyne) in rat SCI. Data are presented as mean±S.E.M., unpaired two-tailed t-test (A-C, E) and one-way ANOVA with Tukey's test (D and F), *p<0.05, **p<0.01, ***p<0.001, ****p<0.0001, n=3-8 in each group.

FIG. 5A-D. Atf3 KO mice had worse neurological outcome in SCI and stroke models. (A) Paw placement and (B) foot drop during grid walk tests showing that Atf3 KO mice had worse functional recovery after SCI compared to WT mice. (C) Sticker removal time from right paw and (D) quantification of left turns in corner test showing that Atf3 KO mice had more severe sensorimotor dysfunction than WT mice 3 days after left pMCAO stroke. Data are presented as mean±S.E.M., n=8-12 in each group. A-B: two-way ANOVA (repeated measures) with Sidak's multiple comparisons; C-D: two-way ANOVA with Tukey's test, *p<0.05, **p<0.01, ****p<0.0001, and “ns” as not significant. WT: wildtype, BL: baseline, CT: control.

FIG. 6A-F. Atf3 KO mice had worse tissue injury after SCI or stroke. (A) Lesion area measured by eriochrome cyanine (EC) staining, the schematic outline, and (B) the quantification of the injury area in spinal cord of WT and Atf3 KO mice 2 weeks after SCI. Scale bar=200 μm. The injury size, presented as the percentage of ipsilateral lesion area in total contralateral uninjured area, was larger in Atf3 KO mice than WT mice 2 weeks post-SCI. (C) Representative images of cresyl-violet stained serial brain sections 3 days after pMCAO and (D) their quantification showing that Atf3 KO mice had larger infarct volume than WT mice. Scale bar=1 mm. (Data are presented as mean±S.E.M., unpaired two tailed t-test. (E) Representative immunohistochemical images of NeuN and FJC peri-infarct ischemia region 3 days after pMCAO and (F) their quantification showing that deletion of Atf3 increased the numbers of FJC⁺ degenerating neurons 3 days after stroke. Scale bar=50 μm. Data are presented as mean±S.E.M., unpaired two-tailed t-test, B: *p<0.05, 4 sections/spinal cord, n=6 or 7 in each group; D: **p<0.001, n=6 in each group; F: **p<0.01, 4 sections/animal, n=6 in each group. Ips: ipsilateral, Cnt: contralatera

FIG. 7A-E. Reduced SPRR1a induction in Atf3 KO mice after SCI or stroke. (A) qRT-PCR results showing the Sprr1a upregulation in spinal cord 1 day post SCI was reduced in Atf3 KO mice compared to WT mice. (B) Western blot quantitative analyses show that the induction of SPRR1a protein in spinal cord 1 day post SCI was reduced in Atf3 KO mice compared to WT mice. (C) Western blot quantitative analyses show that the induction of SPRR1a protein in peri-infarct region 3 days post pMCAO was abolished in Atf3 KO mice. (D) blood SPRR1a levels are significantly elevated 1 day after rat SCI. Data are presented as mean±S.E.M., two-way ANOVA, *p<0.05, **p<0.01, ****p<0.0001, n=3 in each group. (A-C); Unpaired two-tailed t-test, **p<0.01, n=3 in each group (D).

FIG. 8A-C: (A) Human serum ATF3 24 hours post-CSI injury. (B). Human serum ATF3 levels in ischemic and hemorragic stroke patients. (C) Human serum ATF3 levels in patients that had cardiac arrest.

FIG. 9. ATF3 is not expressed in oligodendrocytes in injured hemi-cord 1 day after SCI. Representative IHC staining of ATF3 and Olig2 in injured hemi-cord 1 dpi. There is no ATF3 signals in Olig2⁺ cells (oligodendrocytes) (4 sections/spinal cord, n=5, scale bar 20 μm).

FIG. 10. Illustration of the areas analyzed in peri-infarct ischemic stroke model. Serial brain sections were stained and images from circled peri-infarct ischemia areas were taken that are shown in FIGS. 2C, 2E and 6F. Scale bar=1 mm.

FIG. 11. Atf3 knockout mice lack expression of ATF3 protein. Western blot quantification shows the increased ATF3 protein expression in WT mice 3 days after stroke, as well as no detectable ATF3 protein in the cortex of Atf3 KO mice with or without stroke. Two-tailed unpaired t-test, **p<0.01, n=3 in each group.

### DETAILED DESCRIPTION

### Terminology

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

As used herein, “ATF3” refers to “cyclic AMP-dependent transcription factor ATF-3” (also referred to as “Activating Transcription Factor 3”) that binds to the CAMP response element (CRE). Human protein ATF3 sequences are available under UniProtKB accession number P18847. The term “ATF3” include variants and isoforms (illustrated accession number P18847) encoded by an ATF3 gene. Human Atf3 gene is localized to cytogenetic band 1q32.3 as defined by HGNC, Entrez Gene and Ensembl. A reference Atf3 nucleic acid sequence is available under accession number NC_000001.11 (Homo sapient chromosome 1, GRCh38.p13 Primary Assembly). UniProt assigns human P18847-1 sequence as the canonical sequence in the UniProt entry. The term “ATF3 polypeptide” as used herein refers to any naturally occurring ATF3 polypeptide variant or isoform. A reference human ATF3 isoform 1 cDNA sequence is available under accession numbers NP_001025458 and NP-001665.

The term “blood sample” includes any blood sample, e.g., serum or plasma samples.

The terms “determining,” “assessing,” “assaying,” “measuring” and “detecting” can be used interchangeably and refer to quantitative determinations.

The term “amount” or “level” refers to the quantity of a polypeptide or polynucleotide of interest present in a sample. Such quantity may be expressed as the total quantity of the polypeptide or polynucleotide in the sample, in relative terms, as a concentration of the polypeptide or polynucleotide in the sample, or as a relative quantity compared to a reference value.

The term “protein,” “peptide” or “polypeptide” are used interchangeably herein to refer to a polymer of amino acid residues. In the context of analysis of the levels of proteins encoded by signatures genes, the terms refer to naturally occurring amino acids linked by covalent peptide bonds. In a broader context, the terms can apply to amino acid polymers in which one or more amino acid residue is an artificial amino acid mimetic of a corresponding naturally occurring amino acid and/or the peptide chain comprises a non-naturally occurring bond to link the residues.

As used herein “an ATF3 RNA” measured in accordance with the invention refers to any RNA encoded by an Atf3 gene, including, for example, mRNA, splice variants, unspliced RNA, fragments, or microRNA.

The term “treatment,” “treat,” or “treating” typically refers to a clinical intervention to ameliorate at least one symptom of CNS injury or otherwise slow progression of injury. This includes preventing or slowing symptoms, diminishment of any direct or indirect pathological consequences of injury, amelioration or palliation of the disease state or improved prognosis. In some embodiments, the treatment may increase overall sensory and/or motor neuron function (e.g., by about 5% or greater, about 10% or greater, about 20% or greater, about 25% or greater, about 30% or greater, about 35% or greater, about 40% or greater, about 45% or greater, about 50% or greater, about 55% or greater, about 60% or greater, about 65% or greater, about 70% or greater, about 75% or greater, about 80% or greater, about 85% or greater, about 90% or greater, about 95% or greater, about 96% or greater, about 97% or greater, about 98% or greater, or about 99% or greater). It is understood that treatment does not necessarily refer to a cure or complete restoration of neuronal function. In some embodiments, for example, for a patient that has a low ATF3 score reflecting a less severe SCI, or stroke, such as ischemic stroke, a “treatment” includes active surveillance to monitor the patient for improvement in neuronal function.

The term “recommending” or “suggesting,” in the context of a treatment of a disease, refers to making a suggestion or a recommendation for therapeutic intervention and/or management of the CNS injury that are specifically applicable to the patient.

The term “subject” or “patient” as used herein is intended to include animals. Examples of subjects include mammals, e.g., humans, nonhuman primates, dogs, cows, horses, pigs, sheep, goats, cats, mice, rabbits, rats, and transgenic non-human animals. In preferred embodiments, the subject is a human.

The term “ATF3 score” as used herein refers to a statistically derived value reflecting the severity of CNS injury, e.g., severity of spinal cord injury or severity of a stroke, such as ischemic stroke, that can provide physicians and caregivers valuable diagnostic and prognostic insight. In some instances, the score provides a projected risk of severity of CNS injury, e.g., following a spinal cord injury or a stroke, such as an ischemic stroke. An individual's score can be compared to a reference score or a reference score scale to determine relative severity of spinal cord injury or stroke, e.g., ischemic stroke, or to assist in the selection of therapeutic intervention or management approaches for the CNS injury.

The term “high ATF3 score,” refers to ATF3 polypeptide level or RNA level in a blood or CSF sample having a numerical value that corresponds to severe injury, e.g., in the top percentile range, such as the top tertile (e.g., top 33%) of a range of scores for CNS injury severity, e.g., spinal cord injury or stroke injury, such as ischemic stroke injury. A “low ATF3 score” refers to an ATF3 polypeptide or RNA level in a blood or CSF sample having a numerical value that corresponds to mild injury, e.g., in the bottom percentile range, such as the lowest tertile of a range of scores for CNS injury severity, e.g., spinal cord injury or stroke injury such as ischemic stroke injury.

As used herein, an “AIS grade” refers to the American Spinal Injury Association Impairment Scale (AIS), which is a standardized neurological examination used to assess sensory and motor levels. Grades are assigned as follows:
Grade A: There is no motor or sensory function below the level of injury.
Grade B: Sensory function, but not motor function, is preserved below the neurologic level (the first normal level above the level of injury) and some sensation is preserved in the sacral segments S4 and S5.
Grade C: Motor function is preserved below the neurologic level, but more than half of the key muscles below the neurologic level have a muscle grade less than 3 (i.e., they are not strong enough to move against gravity).
Grade D: Motor function is preserved below the neurologic level, and at least half of the key muscles below the neurologic level have a muscle grade of 3 or more (i.e., the joints can be moved against gravity).
Grade E: All motor and sensory functions are unhindered.
   ATF3 Levels Associated with Severity of Neuronal Injury

Described herein are methods and compositions for classifying severity of CNS injury following a spinal cord injury (SCI) or a stroke, such as an ischemic or hemorrhagic stroke. In some embodiments, CNS injury can result from conditions such as cardiac arrest or other injuries that disrupt circulation to the brain. In one embodiment, method of classifying the severity of CNS injury comprises determining the level of ATF3 protein in a blood sample, e.g., serum, or cerebrospinal fluid (CSF) sample from a SCI patient or from a stroke patient, that has had an ischemic or hemorrhagic stroke. In some embodiments, the level of ATF3 RNA levels circulating in the blood, or present in CSF, following SCI can be measured. In some embodiments, the level of ATF3 RNA levels circulating in the blood, or present in CSF, following a stroke can be measured. In some embodiments, ATF3 polypeptide levels in blood or CSF are determined within hours, e.g., within 24 hours, e.g., from 2 to 24 hours, or 4 to 24, or 6-24 hours, of an SCI. In some embodiments, ATF3 polypeptide levels in blood or CSF are determined within hours, e.g., within 24 hours, e.g., from 2 to 24 hours, or 4 to 24, or 6-24 hours, of cardiac arrest. In some embodiments, ATF3 polypeptide levels are determined from 2 hours up to 1 week of an SCI. In some embodiments, ATF3 polypeptide levels are determined from 2 hours up to 1 week of cardiac arrest. In some embodiments, ATF3 polypeptide levels in blood or CSF are determined within hours, e.g., within 24 hours, e.g., from 2 to 24 hours, or 4 to 24, or 6-24 hours, of a stroke, such as an ischemic stroke; or a hemorraghic stroke. In some embodiments, ATF3 polypeptide levels are determined from 2 hours up to 1 week of an ischemic or hemorrhagic stroke. In some embodiments, RNA levels in the blood or CSF of a subject are determined within 24 hours, e.g., from 1 to 24 hours, of a SCI or of a stroke, or of within 24 hours, e.g., from 1 to 24 hours of cardiac arrest.

Association of ATF3 Levels with Severity of Spinal Cord Injury

The level of ATF3 protein determined in a blood or CSF sample from a subject having an SCI can be transformed into a score that reflects the severity of the injury. For example, in some embodiments, an ATF3 level can be compared to a value associated with SCI clinical severity in a reference population. For example, in some instances, risk of a severe injury is represented by any value in the top tertile of a reference range of ATF3 patients having SCI. In other instances, a severe injury ATF3 score represents values above a threshold calibrated to the top tertile of risk of severe injury. In illustrative embodiments, the severity of injury of a patient having an ATF3 level within 1 standard deviation, or in some instances two standard deviations, of a mean value determined for a reference population of subjects classified as having an AIS A injury is indicative of severe injury; the severity of injury of a patient having an ATF3 level within 1 standard deviation, or in some instance, two standard deviations, of a mean value determined for a reference population of subjects classified as having an AIS B or AIS C injury is indicative of moderate injury; whereas ATF3 levels below the mean value for an AIS B or AIS C injury (plus or minus one standard deviation, or two standard deviations) is classified as mild injury.

Similarly, in some embodiments, RNA levels in blood or CSF are measured and converted into a score that reflect the severity of injury. For example, an ATF3 RNA level can be compared to scores from a reference population, wherein risk of a severe injury is represent by any value in the top tertile of the reference range.

Association of ATF3 Levels Associated with Severity of Stroke

In some embodiments, the level of ATF3 protein determined in a blood sample, e.g., serum, from a subject having a stroke can be transformed into a score that reflects the severity of the stroke. In some embodiments, the stroke is an ischemic stroke. In some embodiments, the stroke is hemorrhagic stroke. For example, in some embodiments, an ATF3 level can be compared to values associated with stroke severity in a reference population. For example, in some instances, a severe stroke is represented by any value in the top tertile of a reference range of ATF3 patients who have had a stroke. In other instances, a severe stroke may represent values above a threshold calibrated to the top tertile of risk of severe injury. In illustrative embodiments, the severity of the stroke in a patient having a blood ATF3 level within one standard deviation, or in some instances two standard deviations, of a mean value determined for a reference population of ischemic stroke subjects classified as having a severe stroke; is indicative of a poor prognosis.

In some embodiments ATF3 RNA levels are determined in a stroke patient and converted into a score that indicates the severity of the stroke. In some embodiments, the stroke is ischemic stroke. In some embodiments, the stroke is hemorrhagic stroke.

### ATF3 Levels

The methods described herein are based, in part, on the identification of ATF3 as a biomarker indicative of the risk of severe CNS injury, such as severe spinal cord injury or injury following a stroke. The level of ATF3 polypeptide or RNA in a blood or CSF sample, or level ATF3 RNA or polypeptide in a CNS tissue sample, e.g., spinal cord sample from an SCI patient, reflects the severity of injury. In some instances, a high risk of severe spinal cord injury may represent values above a threshold calibrated to the top tertile of levels of ATF3 in blood or spinal fluid that correlates with severe spinal cord injury. In some instances, a high risk of severe injury from stroke may represent values above a threshold calibrated to the top tertile of levels of ATF3 in blood or spinal fluid that correlates with severe stroke injury.

In a further aspect, the disclosure provides a method of processing a blood or CSF sample from a patient, the method comprising evaluating the level of ATF3 protein in the blood or CSF sample obtained from a subject having a CNS injury, such as a SCI patient or stroke patient, shortly after injury, e.g., within 24 hours; and quantifying levels of protein, compared to a reference score or a reference score scale obtained from analysis of ATF3 levels in the blood of patients having an SCI injury, or for stroke patients, e.g., ischemic stroke patients. In some embodiments, the step of quantifying the level of ATF3 polypeptide comprises performing an immunological assay, such as ELISA, that employed an anti-ATF3 antibody. In some embodiments, the level of ATF3 polypeptide may be assessed using a binding agent such as an aptamer.

In some embodiments, a blood of CSF sample from an SCI patient or stroke patient, e.g., ischemic stroke patient, is processed and the level of ATF3 RNA determined. The level can be compared to a reference scores scale obtained form analysis of ATF3 RNA in blood from a corresponding patient population.

### Methods of Determining ATF3 Protein Levels

In some embodiments, methods of determining levels of ATF3 in a subject having an SCI comprises determining the level of ATF3 levels in a blood or CSF sample from the subject, e.g., obtained within 24 hours of the SCI.

In some embodiments, methods of determining levels of ATF3 in a subject having an ischemic stroke comprises determining the level of ATF3 levels in a blood or CSF sample from the subject, e.g., obtained within 24 hours of the stroke.

In some embodiments, ATF3 polypeptide levels are determined using an immunoassay, such as a sandwich immunoassay, competitive immunoassay, and the like. Thus, for example, an anti-ATF3 antibody may be employed for assessing protein levels in a blood or CSF sample. In some embodiments, ATF3 polypeptide levels may be determined using mass spectrometry methods or by electrophoretic methods.

In some embodiments, the level of ATF3 polypeptide can be normalized to a reference level for one or more control proteins. In some embodiments, the normalized amount of protein may be compared to the amount found in an SCI reference set (for an SCI patient sample) or an ischemic stroke reference sent (for a stroke patient). A control value can be predetermined, determined concurrently, or determined after a sample is obtained from the subject. Thus, for example, the reference control level for normalization can be evaluated in the same assay or can be a known control from a previous assay.

### Methods of Quantifying ATF3 RNA

In some embodiments, methods of the present disclosure comprise detecting the level of RNA expression, e.g., mRNA expression, of ATF3 present in a blood sample, e.g., serum, or a CSF sample, from a subject that has a SCI, e.g., within 24 hours of injury.

In some embodiments, methods of the present disclosure comprise detecting the level of RNA expression, e.g., mRNA expression, of ATF3 present in a blood sample, e.g., serum, or a CSF sample, from a subject that had a stroke, e.g., an ischemic stroke, e.g., within 24 hours of the stroke.

The level of RNA (e.g., mRNA) can be detected or measured by a variety of methods including, but not limited to, an amplification assay, a hybridization assay, or a sequencing assay. Non-limiting examples of such methods include quantitative RT-PCR, quantitative real-time PCR (qRT-PCR), digital PCR, nanostring technologies, ligation chain reaction, in situ hybridization, oligonucleotide elongation assays, mass spectroscopy, and cDNA-mediated annealing, selection, extension, and ligation and the like. In some embodiments, expression level is determined by sequencing, e.g., using massively parallel sequencing methodologies. For example, RNA-Seq can be employed to determine RNA expression levels.

The level of mRNA can be normalized to a reference level for one or more control genes. In some embodiments where the CNS injury is a spinal cord injury, the normalized amount of RNA may be compared to the amount found in an SCI reference set. In some embodiments where the CNS injury is an ischemic stroke, the normalized amount of RNA may be compared to the amount found in an ischemic stroke reference set. A control value can be predetermined, determined concurrently, or determined after a sample is obtained from the subject. Thus, for example, the reference control level for normalization can be evaluated in the same assay or can be a known control from a previous assay.

### Establishing CNS Injury Severity Scores Based on ATF3

After determining the level of ATF3 polypeptide in a blood or CSF sample, or ATF3 RNA level, the method presented herein includes calculating an ATF3 score, e.g., which is indicative of the severity of the CNS injury, e.g., spinal cord injury stroke injury, e.g., ischemic stroke injury.

The term “reference value” in the context of the present invention is to be understood as a predefined level of ATF3 polypeptide, or RNA, in a sample or group of samples. The reference value can be an absolute value; a relative value; a value that has an upper or a lower limit, a range of values; an average value; a median value, a mean value, or a value as compared to a particular control or baseline value. Methods for obtaining the reference value from the group of subjects selected are well known in the state of the art.

In some embodiments, an SCI subject's ATF3 score is categorized as “high,” “intermediate,” or “low” relative to a reference scale, e.g., a range of ATF3 scores from a population of reference SCI subjects. In some embodiments, an ischemic stroke subject's ATF3 score is categorized as “high,” “intermediate,” or “low” relative to a reference scale, e.g., a range of ATF3 scores from a population of reference ischemic stroke subjects. In some cases, a high score, indicative of more severe injury, corresponds to a numerical value in the top tertile (e.g., the highest ⅓) of the reference scale; an intermediate score corresponds to the intermediate tertile (e.g., the middle ⅓) of the reference scale; and a low score corresponds to the bottom tertile (e.g., the lowest ⅓) of the reference scale. In other embodiments, a high score represents an ATF3 score that is 0.66 or above, e.g., 0.66, 0.67, 0.70, 0.75, 0.80, 0.85, 0.90, 0.95, 0.99 or 1.0 based on a normalized, standardized reference scale on a scale of 0 to 1. In other embodiments, a subject's ATF3 score is compared to one or more threshold value(s) to provide a likelihood of severe injury. In some cases, the high ATF3 score corresponds to a numerical value, e.g., a risk score in the top 5%, top 10%, top 15%, top 20%, top 25%, top 30%, top 35%, top 40%, top 45%, top 50%, or top 60% of the reference scale. In some cases, the high ATF3 score corresponds to a numerical value, e.g., a risk score in the top 5%, top 10%, top 15%, top 20%, top 25%, top 30%, top 35%, top 40%, top 45%, or top 50% of the reference scale. In some cases, the high ATF3 score corresponds to a numerical value, e.g., a risk score in the top 5%, top 10%, top 15%, top 20%, top 25%, top 30%, top 35%, or top 40% of the reference scale.

As noted above, in order to establish a reference ATF3 scale or a threshold value for practicing the method of this invention, a reference population of subjects having a SCI, can be used to establish a range of ATF3 values associated with severity of the injury. Thus, in for an SCI injury, a reference ATF3 scale or a threshold value for practicing the method of this invention is established in a reference population of subjects having SCI, which can be used to establish a range of ATF3 values associated with the grade of spinal cord injury. In some embodiments, the reference population may have the type of SCI as the test subject. In some embodiments the reference scale is a plurality of ATF3 scores derived from analysis of spinal cord injuries from a population of reference subjects. Optionally, the reference subjects may be of the same gender or similar age.

For stroke injury, e.g., ischemic stroke injury, or in alternative embodiments, hemorraghic stroke injury, a reference ATF3 scale or a threshold value for practicing the methods of the present disclosure established in a reference population of subjects that have had a stroke can be used to establish a range of ATF3 values associated with the severity of injury due to the ischemic stroke. In some embodiments the reference scale is a plurality of ATF3 scores derived from analysis of stroke from a population of reference subjects. Optionally, the reference subjects are of same gender, similar age, or similar ethnic background. In some embodiments, the reference subjects have the same type of stroke as the patient, e.g., the reference scale and/or reference values used for an ischemic stroke patient are from a reference population of ischemic stroke patients. Similarly, in some embodiments, the reference scale and/or reference values used for a hemorrhagic stroke patient are from a reference population of hemorrhagic stroke patients.

An analysis similar to those above can be performed for patients that have suffered from cardiac arrest.

An ATF3 score may be used in decision-making regarding therapeutic treatment. For example, based on the score, a clinician can initiate treatment without delay such as decompressive surgery to release the compression on spinal cord tissue. Other treatments, such as vasopressor use to control patient's blood pressure and maintain spinal cord perfusion, ventilation machine use for SCI patients with respiratory function compromise, will depend on the severity of the injury. Patients with more severe injury will need these treatments for longer period of time.

In some embodiments, a subject, e.g., with intermediate or severe injury, may be treated with SPRR1a protein delivered locally, e.g., by intrathecal injection, or intravenously. In some embodiments, the protein may be provided via methodology in which a nucleic acid encoding the protein is introduced, e.g., intrathecally. Such a nucleic acid may be RNA or DNA, including plasmids, viral vectors, and the like.

### Computer-Implemented Methods, Systems, and Devices

Any of the methods described herein may be totally or partially performed with a computer system including one or more processors, which can be configured to perform the steps. Thus, embodiments are directed to computer systems configured to perform the steps of any of the methods described herein, potentially with different components performing a respective step or a respective group of steps. Although presented as numbered steps, steps of methods herein can be performed at a same time or in a different order. Additionally, portions of these steps may be used with portions of other steps from other methods. Also, all or portions of a step may be optional. Any of the steps of any of the methods can be performed with modules, circuits, or other means for performing these steps.

Any of the computer systems mentioned herein may utilize any suitable number of subsystems. In some embodiments, a computer system includes a single computer apparatus, where the subsystems can be the components of the computer apparatus. In other embodiments, a computer system can include multiple computer apparatuses, each being a subsystem, with internal components. For example, in some embodiments, a computer system may include storage device(s), a monitor coupled to a display adapter, and a keyboard. Peripherals and input/output (I/O) devices, which couple to an I/O controller, can be connected to the computer system by any number of means known in the art, such as a serial port. For example, a serial port or external interface (e.g. Ethernet, Wi-Fi, etc.) can be used to connect a computer system to a wide area network such as the Internet, a mouse input device, or a scanner. The interconnection via a system bus allows the central processor to communicate with each subsystem and to control the execution of instructions from system memory or the storage device(s) (e.g., a fixed disk, such as a hard drive or optical disk), as well as the exchange of information between subsystems. The system memory and/or the storage device(s) may embody a computer readable medium. Any of the data mentioned herein can be output from one component to another component and can be output to the user.

A computer system can include a plurality of the same components or subsystems, e.g., connected together by external interface or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

It should be understood that any of the embodiments of the present disclosure can be implemented in the form of control logic using hardware (e.g., an application specific integrated circuit or field programmable gate array) and/or using computer software with a generally programmable processor in a modular or integrated manner. As user herein, a processor includes a multi-core processor on a same integrated chip, or multiple processing units on a single circuit board or networked. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C++ or Perl using, for example, conventional or object-oriented techniques. The software code may be stored as a series of instructions or commands on a computer readable medium for storage and/or transmission, suitable media include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium according to an embodiment of the present invention may be created using a data signal encoded with such programs. Computer readable media encoded with the program code may be packaged with a compatible device or provided separately from other devices (e.g., via Internet download). Any such computer readable medium may reside on or within a single computer product (e.g., a hard drive, a CD, or an entire computer system), and may be present on or within different computer products within a system or network. A computer system may include a monitor, printer, or other suitable display for providing any of the results mentioned herein to a user.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

### I. EXAMPLES

The following examples are offered to illustrate, but not to limit, the claimed invention. The Examples describe the identification and validation of ATF3 as an indicator of spinal cord injury.

### Example 1. Identification of ATF3 as an Indicator of CNS Injury

To identify a new biomarker that is expressed mainly in the injured CNS neurons, we performed RNA sequencing (RNA-Seq) in injured mouse spinal cord and found that activating transcription factor 3 (Atf3), a gene that is induced only in the injured peripheral sensory neurons, was one of the most significantly upregulated genes 4 hours after spinal cord injury (SCI). Quantitative RT-PCR confirmed the upregulation of Atf3 gene in the injured spinal cord tissues shortly after SCI, and western blot showed that ATF3 protein level was also increased in the ischemic mouse brain. Histological study demonstrated ATF3 protein was induced specifically in injured neurons 1 day after SCI or ischemic stroke. Importantly, ATF3 protein levels in mouse blood and cerebrospinal fluid (CSF) were detectable and elevated after SCI and stroke, suggesting ATF3 can be a specific biomarker for detecting neuronal injuries in acute CNS injuries. Compared to WT control, Atf3 KO mice had worse functional recovery, enlarged injury areas, more damaged neurons and reduced regenerative responses after SCI or stroke, indicating that ATF3 has a neuroprotective role. Thus, ATF3 is a biomarker for detecting neuronal damage after acute CNS injuries and has a neuroprotective function. Experimental description is detailed below.

Atf3 Gene was Upregulated in Spinal Cord Tissue Shortly after SCI

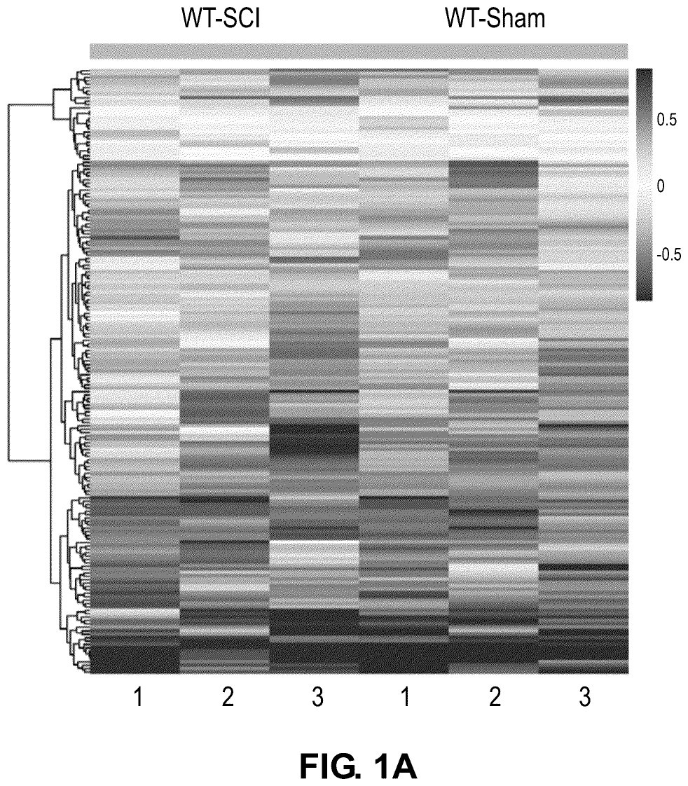

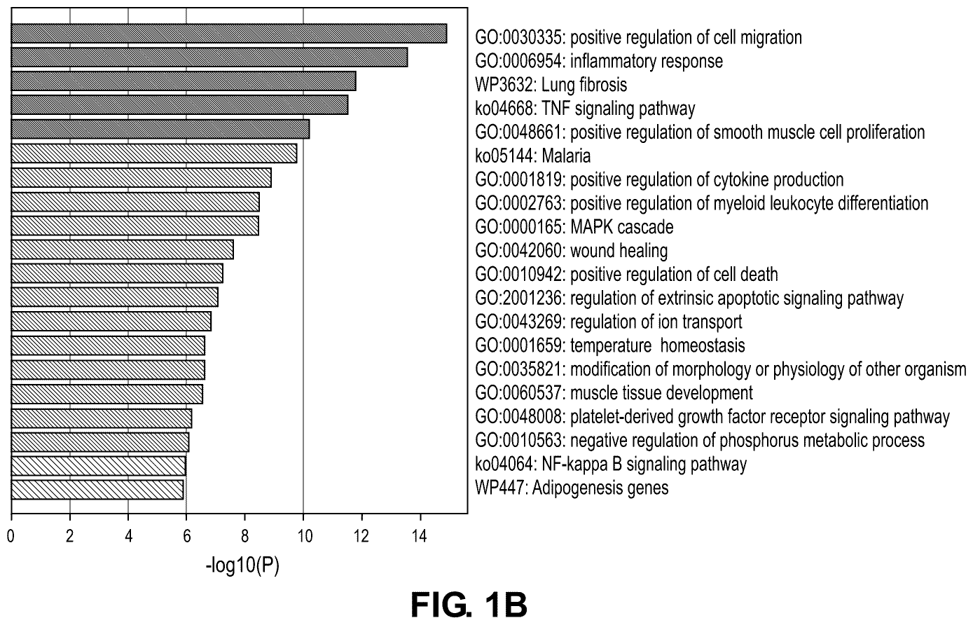

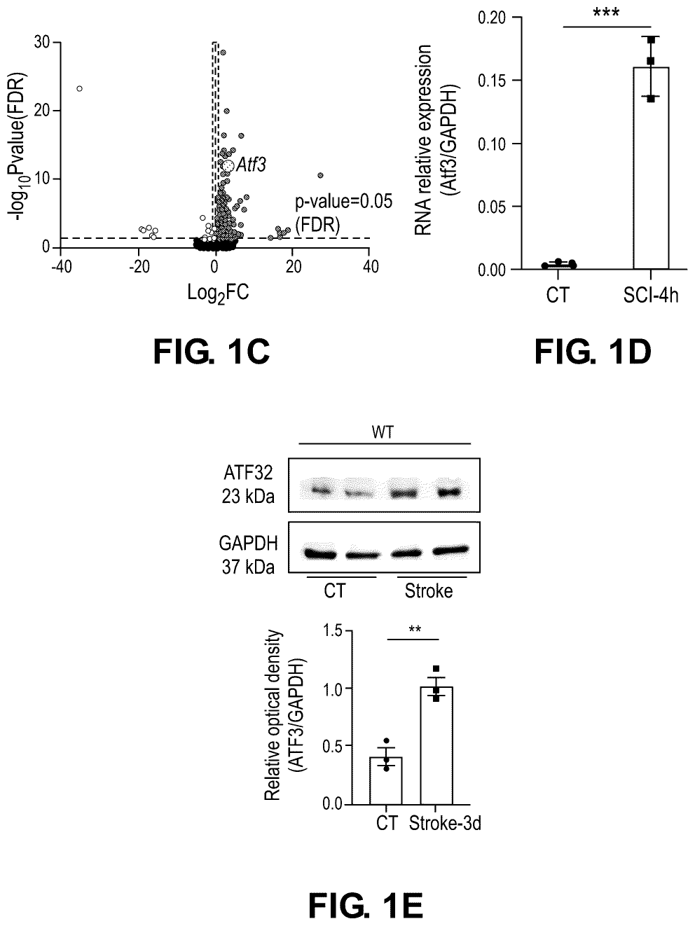

To identify potential biomarkers of injured CNS neurons, we did RNA sequencing (RNA-Seq) of mouse spinal cord tissues before and shortly after SCI. Unlike the previous RNA-Seq studies that examined the gene expression profiles of injured spinal tissue at later timepoints after SCI (13-15), we analyzed gene expression profiles 4 hours after SCI because we aimed to discover the new biomarkers in the early phase of CNS injuries, because the early timepoints are crucial periods for the development of pathophysiological changes in CNS injuries when many injured neurons are likely still alive and can be rescued. Compared to sham control, injured spinal cord had a total of 177 genes with more than 1.5-fold changes (either up- or down-regulated) with multiple comparisons (Benjamini-Hochberg false discovery rate (16); BHFDR p<0.05). Among the differentially expressed genes (DEG), 160 genes were upregulated and 17 were downregulated (FIG. 1A). The major pathways identified by gene ontology (GO) analysis included inflammatory responses, cellular apoptotic responses, signaling transduction pathways, which all are the acute reactions to trauma in spinal tissue (FIG. 1B). We found that Atf3 was one of the most upregulated genes, with 9.9-fold increase detected by RNA-Seq (Volcano plot in FIG. 1C). This is intriguing because it has been reported that Atf3 is upregulated only in the injured peripheral dorsal root ganglion (DRG) sensory neurons after peripheral nerve injury. Our quantitative RT-PCR confirmed that Atf3 expression in spinal cord was low in sham controls, but was dramatically upregulated (by 25 folds) 4 hours after SCI (FIG. 1D). In addition, in a mouse ischemic stroke model created by occluding the middle cerebral artery permanently (pMCAO) the Atf3 protein levels were significantly high in ischemic brain tissues than the corresponding tissues collected from the sham controls (FIG. 1E-F).

ATF3 Protein was Induced Specifically in CNS Neurons after Injury

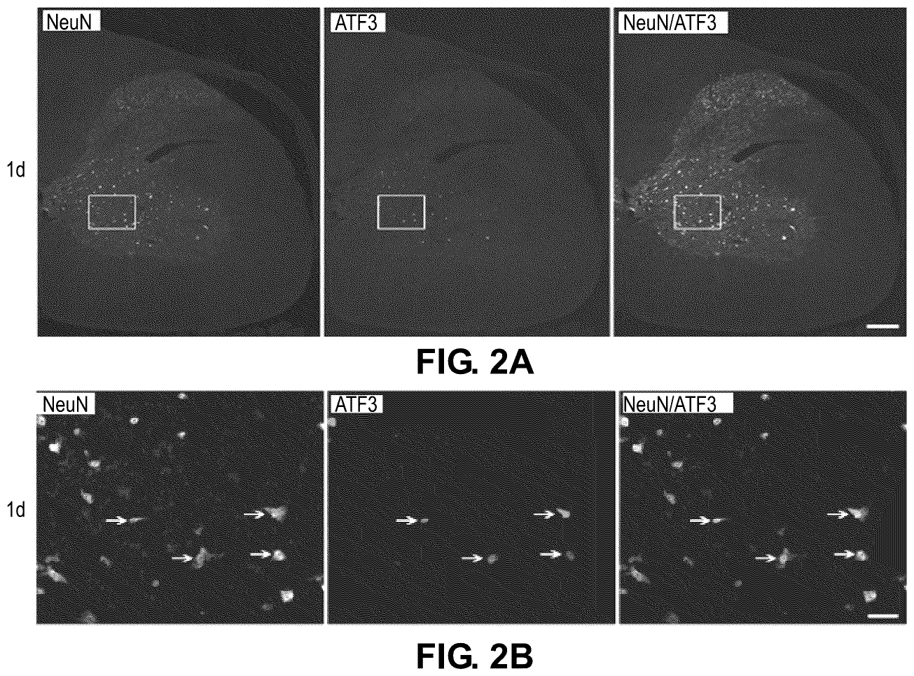

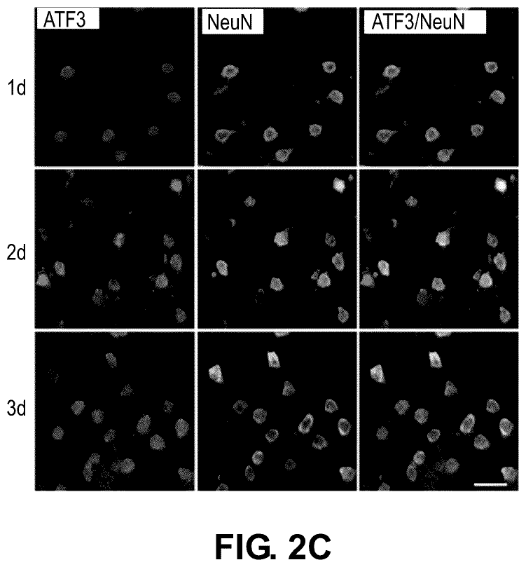

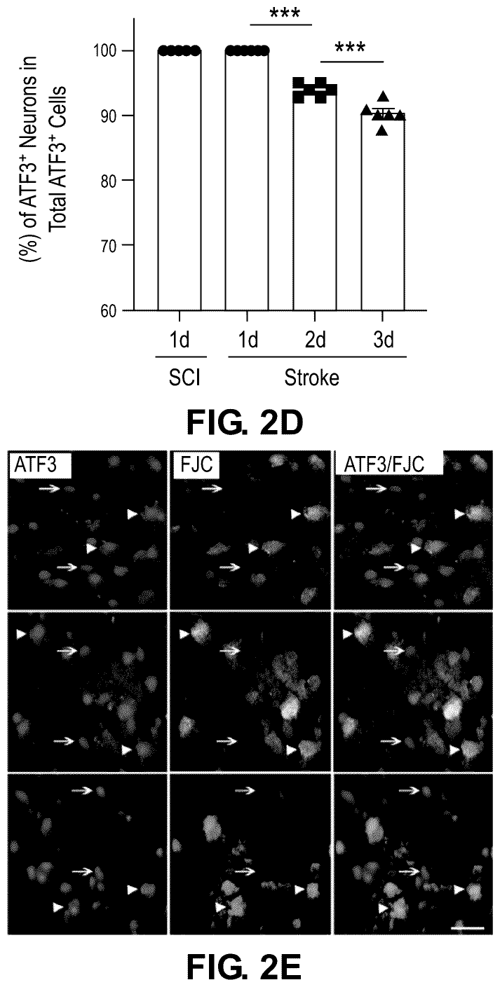

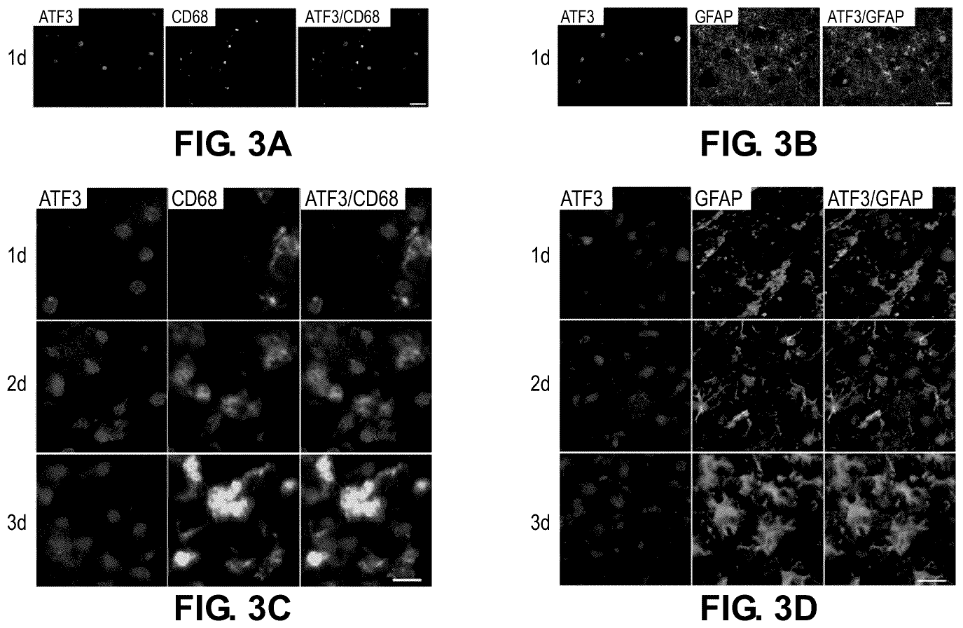

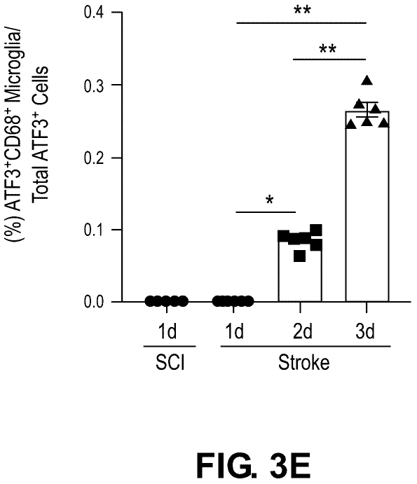

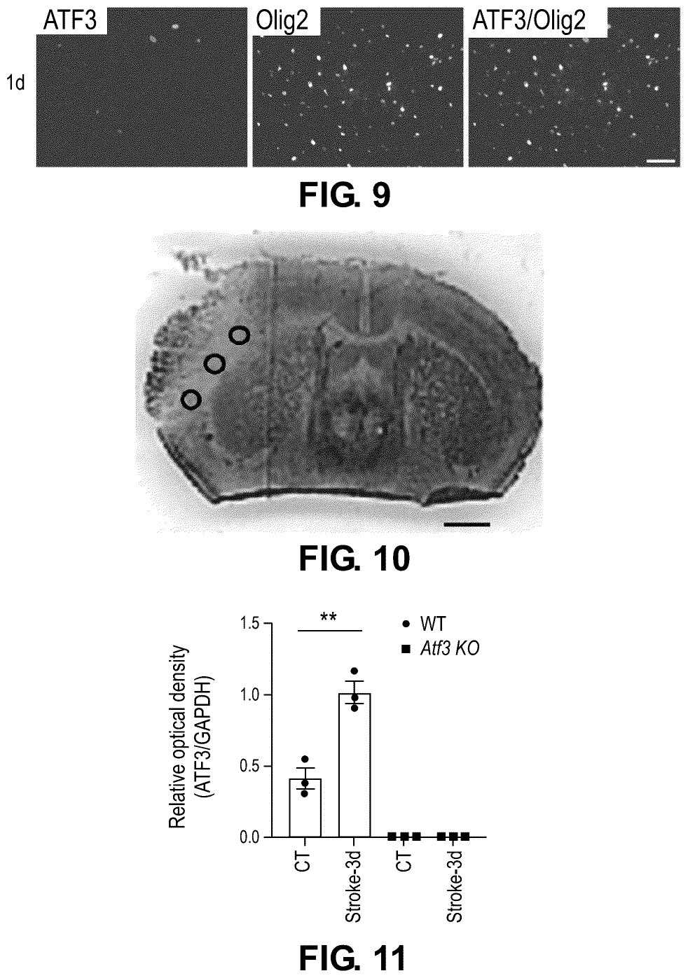

As ATF3 is widely considered as a cellular marker of injured sensory neurons after peripheral nerve injury, we wondered if ATF3 is also a cellular marker of injured CNS neurons. We therefore performed immunohistochemical (IHC) staining to investigate the cell types that expressed ATF3 in the injured spinal cord and ischemic stroke brain. Using a rat model of unilateral cervical SCI, we found the ATF3 protein was only present in the NeuN⁺ neurons of injured spinal cord (FIGS. 2A and 2B), without detectable ATF3 signals in microglia (CD68⁺, FIG. 3A), astrocytes (GFAP⁺, FIG. 3D) or oligodendrocytes (Olig2*, (FIG. 9) 1 day post injury (dpi). Similarly, in the ischemic brain tissue of mouse pMCAO model (FIG. 2C), ATF3 was also exclusively expressed in NeuN⁺ neurons (FIG. 2D), but not in CD68⁺ microglia or GAFP⁺ astrocytes (FIGS. 3B and 3E) 1 day after stroke, although a small number of non-neuronal cells started to express ATF3 2 to 3 days later (FIG. 2E, 3C). By co-immunostaining ATF3 with Fluoro-Jade C (FJC), a known marker for degenerated neurons (17) of cortex after stroke, we found that all the FJC⁺ cells were ATF3⁺, but many ATF3⁺ cells were FJC-(FIG. 2F). Interestingly, most ATF3⁺/FJC⁺ cells have enlarged nucleus with irregular shape, consistent with the fact that FJC specifically label the degenerating neurons. On the other hand, most ATF3⁺/FJC⁻ cells have small and regular nucleus, indicating that these cells are at the early stage of injury but not yet degenerated (FIG. 2F). These results suggest that ATF3 is a cellular marker for injured neurons that are degenerating or at pre-degenerating stage after CNS injuries. Of note, FJC has massive nonspecific staining in spinal cord tissue after SCI (data not shown) and thus cannot be used to label the degenerating neurons in SCI, further indicating the superiority of ATF3 to FJC in labelling the injured neurons.

### ATF3 is a Biomarker for Acute CNS Injury

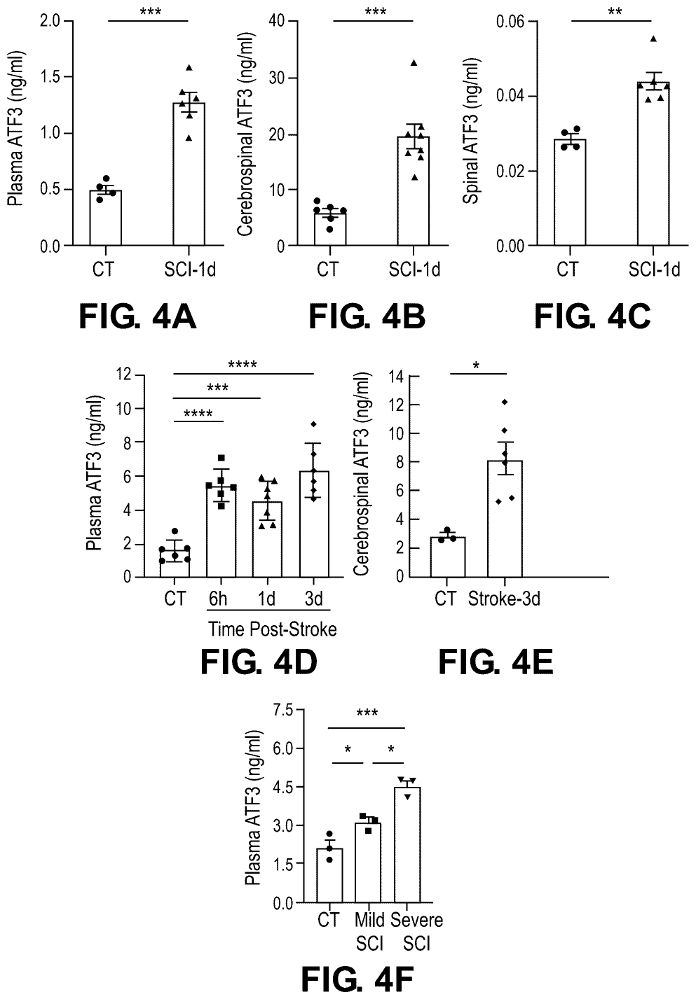

As ATF3 was induced exclusively in injured neurons 1 day after SCI and ischemic stroke, we explored the possibility to detect ATF3 protein levels in peripheral blood and cerebrospinal fluid (CSF) as a biomarker for acute CNS injuries. Mouse blood, CSF and spinal cord tissue were collected 1 day after sham surgery or SCI and analyzed via ELISA. Compared to sham control, mice subjected to SCI has higher level of ATF3 protein in the blood, CSF and spinal tissue (FIG. 4A-C). Similarly, the ATF3 protein levels were increased in plasma 6 hours, 1 day, and 3 days after pMCAO (FIG. 4D), as well as in CSF 3 days after pMCAO (FIG. 4E). Thus, ATF3 can be measured as a new biomarker in blood in clinical to detect neuronal injury at acute stage after CNS injuries.

Atf3 KO Mice have Impaired Neurological Function after SCI and Ischemic Stroke

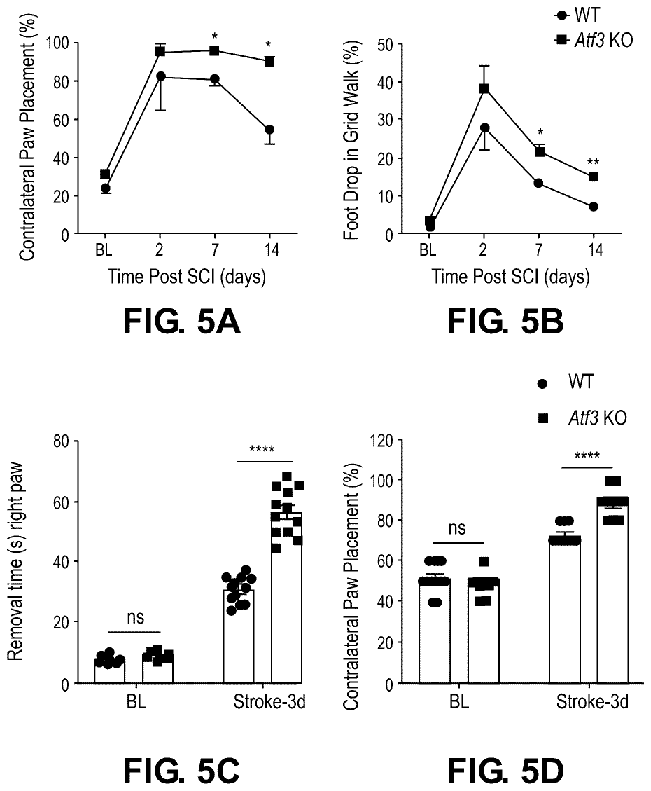

To investigate the functional role of ATF3 in CNS injures, we assessed and compared the functional recovery in WT and Atf3 KO mice (18) with a series of behavioral tests after SCI and stroke. With western blot we confirmed that ATF3 protein were not detectable in the brain of Atf3 KO mice with or without stroke (FIG. 5A-B). Similarly, with qRT-PCR we also confirmed that Atf3 gene is not expressed in the spinal cord of Atf3 KO mice (data not shown). With paw placement test (19) to investigate the motor function recovery after SCI, we observed that Atf3 KO mice had significantly less ipsilateral weight support (increased contralateral uninjured forepaw placement against cylinder for weight support) compared to WT animals over 2 weeks (FIG. 5C), indicating that the Atf3 KO mice had worse motor dysfunction after SCI than WT mice. In addition, by the foot drop test during grid walk (20), we found that the Atf3 KO mice, compared to WT mice, demonstrated increased foot drops while walking on the grid after SCI (FIG. 5D), further supporting that Atf3 KO mice had impaired functional recovery from SCI.

For stroke mice, we performed adhesive removal (21) and corner tests (22) in both WT and Atf3 KO mice before and 3 days after left pMCAO to investigate the sensorimotor function. In adhesive removal test, we found that the WT and the Atf3 KO mice spent similar time to remove sticker from their right paws at the baseline before pMCAO (FIG. 5E). Although both WT and Atf3 KO mice took longer time to remover sticker from their right paws 3 days after pMCAO, the Atf3 KO mice spent significantly longer time than WT mice to remove stickers from their right paws (FIG. 5E), suggesting that the Atf3 KO mice had more severe sensorimotor dysfunction after stroke than WT mice. In corner test, both WT and Atf3 KO mice made about 50% of left turns (ipsilateral side of stroke) at baseline before stroke (FIG. 5F). Although both WT and Atf3 KO mice made more left turns 3 days after left pMCAO compared to baseline, Atf3 KO mice made significantly more left turns than WT mice (FIG. 5F), further supporting that knockout Atf3 exacerbates sensorimotor dysfunction of stroke mice.

### Atf3 KO Mice Exhibited More Severe CNS Tissue Damage and Neuronal Degeneration

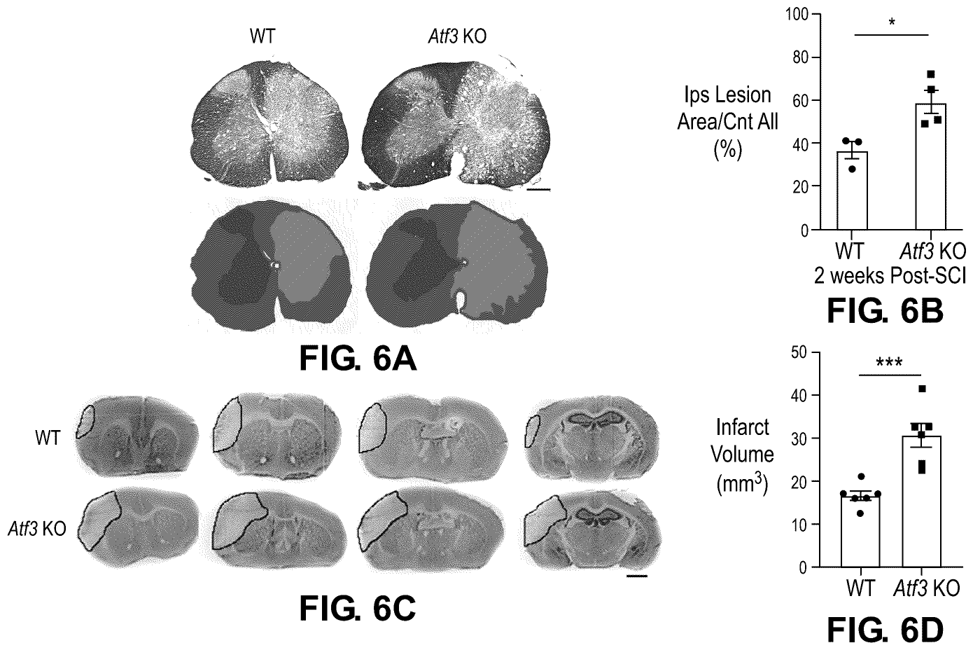

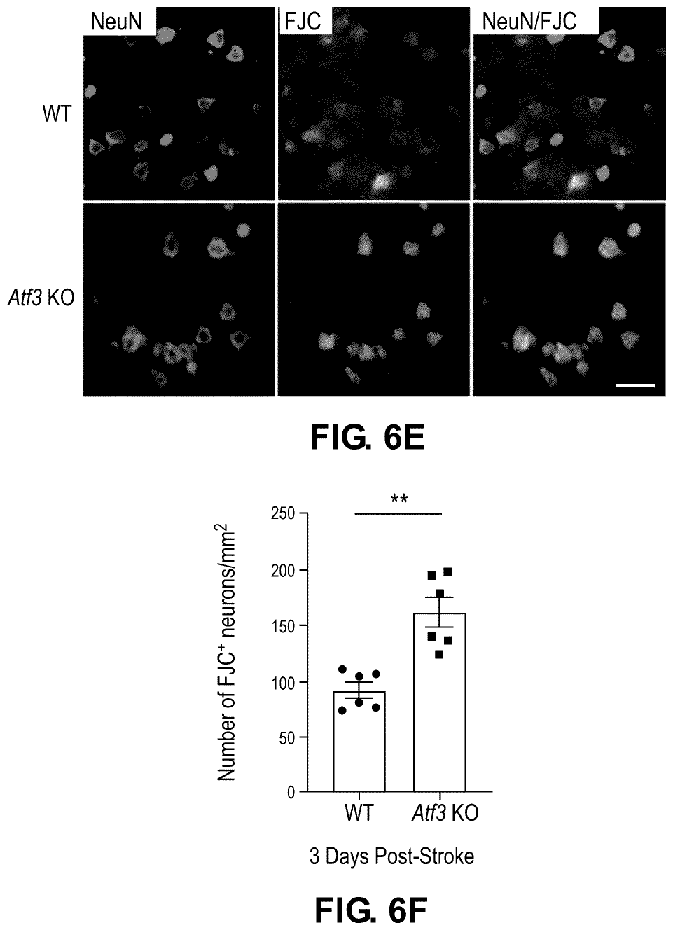

To further explore the effect of ATF3 in CNS injuries, we examined the lesion size after SCI or pMCAO in both WT and Atf3 KO mice. We evaluated the maximal lesion area at the injury epicenter 2 weeks after SCI with eriochrome cyanine (EC) staining (23), and found that the ratio of ipsilateral lesion area to total contralateral uninjured area is significantly enlarged in Atf3 KO, compared to WT mice (FIG. 6A-B). We also measured and compared infarct volumes in the brains collected 3 days after pMCAO and found that the Atf3 KO mice had significantly larger infarct volumes than WT mice (FIGS. 6C and 6D). We further demonstrated that Atf3 KO mice had significant more degenerating neurons (FJC⁺) than WT mice 3 days after pMCAO in the peri-infarct region after pMCAO (FIG. 6E-F). These data indicate that Atf3 KO mice have more severe neuronal damage than WT mice after SCI and stroke. Taken together, these results suggest that ATF3 has neuroprotective function after CNS injuries.

Atf3 Deficiency Diminishes Neural Regenerative Responses after CNS Injuries

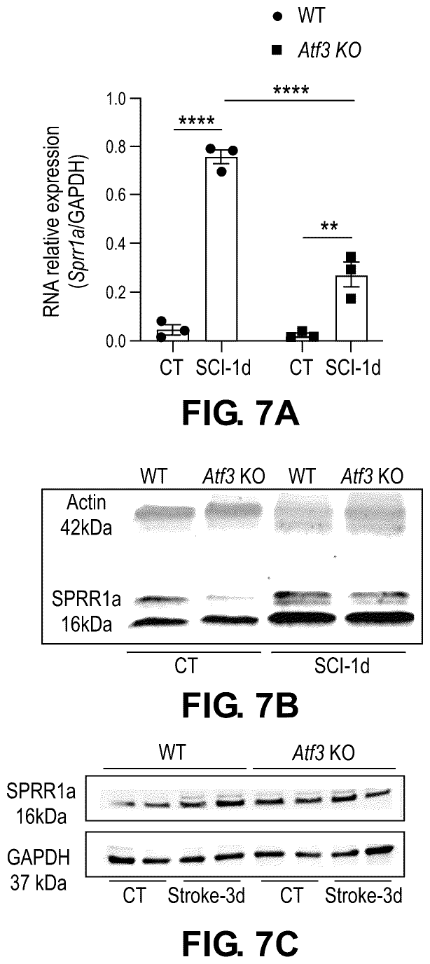

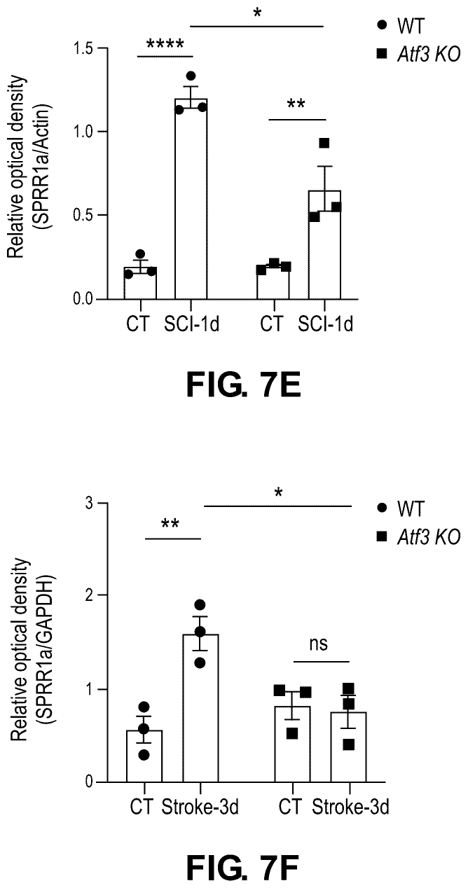

To investigate the mechanism of neuroprotective effect of ATF3 after CNS injuries, we examined the expression of Sprr1a (small proline-rich repeat protein 1a), a regeneration-associated gene (RAG) that is critical for axonal regeneration after neuronal injury (24). With qRT-PCR we analyzed the expression of Sprr1a gene was upregulated in the spinal code of WT mice 1 day after SCI. The upregulation of Sprr1a expression was significantly reduced in Atf3 KO mice (FIG. 7A). With Western blot we also found that SPRR1a protein levels were elevated in the spinal cord or the brain after SCI or pMCAO. The elevations in both injury models were significantly reduced in Atf3 KO mice (FIG. 7B-C). Further, mouse SPRR1a protein could be detected in blood by ELISA and it was also elevated 24 hours after SCI (FIG. 7D). These data indicate that ATF3 has a neuroprotective function after SCI and pMCAO, which is likely through promoting the expression of regeneration-associated genes such as Sprr1a. SPRR1a protein may also serve as part of a biomarker signature of SCI.

### Analysis of Experimental Results of Example 1

In the current study, we demonstrate that the transcription factor ATF3, a member of the ATF/cyclic AMP response element-binding (CREB) family of transcription factors (25), is induced specifically in CNS neurons shortly after SCI and ischemic stroke. Importantly, ATF3 protein is detectable and elevated in the blood and CSF shortly after the injuries, suggesting that ATF3 could serve as a biomarker to detect neuronal injury at the acute stage of these injuries, which may assist clinicians stratify patients based on injury severity, design therapeutic strategies and prepare patients for clinical trials, monitor patient recovery and response to treatment. Further, ATF3 also has a neuroprotective function after CNS injuries.

Injuries to spinal cord and brain can be devastating to cause significant functional disabilities, including permanent sensorimotor dysfunctions and cognitive impairment (26-29). One of the major pathologies in CNS injury is neuronal damage and death (30-34), which are highly correlates with injury severity and functional outcome. However, identification of specific cellular markers for neuronal injury has not been completely satisfactory. FJC (17, 35-37) has been widely used to mark degenerating neurons, although it is unclear if FJC can label the injured neurons that have not been degenerated. Indeed, FJC has never been reported to label the injured but not degenerated sensory neurons after peripheral nerve injury. Caspases (30, 38) and TUNEL (terminal deoxynucleotidyl transferase dUTP nick end labeling) (39) stains are also commonly used to label the neurons that undergo programmed cell death, but they are neither neuron specific, nor effective in detecting the injured neurons that are not dying. However, unlike those degenerated or dying neurons, the early-stage injured neurons are more likely to recover from the injury and thus should be the main cellular targets for intervention.

The importance of cellular marker for injured neurons in understanding how neurons respond to injury has been clearly illustrated in the model of peripheral nerve injury. Atf3 is induced in DRG sensory neurons after peripheral nerve injury and is widely recognized as a cellular marker of injured sensory neurons (25, 40, 41). This knowledge has remarkably facilitated the study on how sensory neurons respond to nerve injury. Using Atf3 as a marker gene for injured DRG neurons, recent single cell RNA-sequencing analysis of DRG neurons has revealed the time course of gene expression profile change in the injured DRG neurons and the contribution of Atf3 in the regulation of gene expression in DRG sensory neurons (42).

Our current study demonstrates the role of ATF3 as a specific cellular marker for injured CNS neurons, because ATF3 is induced exclusively in injured spinal cord neurons 1 day post SCI and in injured brain neurons 1 day after ischemic stroke. Interestingly, we found that after stroke all FJC⁺ neurons were ATF3⁺, but many ATF3⁺ neurons were FJC⁻, suggesting that ATF3 is more sensitive than FJC to detect injured neurons. In fact, unlike the ATF3⁺/FJC⁺ cells that have enlarged irregular nucleus, the ATF3⁺/FJC-cells have relatively small and regular nucleus (FIG. 2F). As FJC is specific to detect degenerated neurons (17, 36), this result indicates that the ATF3⁺/FJC-cells are likely the injured neurons that are not in the process of neurodegeneration yet, and thus these neurons can likely be saved by therapeutic interventions. Further study of ATF3⁺ neurons will likely help us to understand how injured CNS neurons react after injury, and to discover and develop the appropriate intervention to save the injured CNS neurons.

Biomarkers for CNS injuries have been extensively studied. For both SCI and TBI, there are promising molecules as biomarker which are cell type or tissue specific (e.g., GFAP for glial cells or NF for axons) or can detect specific pathology after injury (bleeding after mild TBI or concussion) (8, 10). In stroke, many biomarker candidates are used to differentiate ischemic and hemorrhagic strokes in helping clinical decisions on whether the patient should receive thrombolytic treatment or embolectomy based on stroke onset time and occlusion location if the patient has ischemic stroke. However, these current biomarkers are proteins either not expressed in the neurons, or expressed nonspecifically in all neurons, and none of these biomarkers are expressed specifically in the injured neurons. It is beneficial to have a biomarker that is mainly expressed in the injured neurons, because such biomarker should have higher sensitivity and specificity for detecting the severity of neuronal injuries, which could potentially correlate better with functional outcome. In this study, we found that ATF3 expression is induced in the injured neurons quickly after CNS injuries, and that ATF3 protein is detectable and elevated in extracellular compartments (CSF and blood) shortly after SCI and stroke (FIG. 4A-E). Thus, the ATF3 protein level in peripheral blood and CSF can be utilized as a new biomarker for CNS injuries that might correlate better with neuronal injury after SCI and stroke.

As a member of CAMP response element binding (CREB) family transcriptional factor, Atf3 has been shown to be protective in peripheral nervous system because Atf3 promotes neurite outgrowth in axotomized cultured DRG neurons (43) and axonal regeneration after peripheral nerve injury (44). In different preclinical models of CNS injuries, the role of Atf3 has also been studied. In a spinal cord transection model in Zebrafish, knock down of Atf3 expression by anti-sense Atf3 morpholino led to decreased swimming distance and less axonal regrowth, compared to control (45), and in mouse traumatic brain injury (TBI), global Atf3 KO mice developed more prominent cerebral hemorrhage (46). In transient ischemic stroke models, Atf3 is upregulated and lentiviral overexpression of Atf3 in cultured murine neurons leads to reduced glutamate neurotoxicity (47), and knockout of Atf3 gene in mice led to more inflammatory responses, exacerbated infarct volume and worse neurological function (48). Further, in a transgenic mouse model of amyotrophic lateral sclerosis (ALS), enhanced Atf3 gene expression in motor neurons increases the survival of injured motor neurons (49). In this study, we found that the FJC⁺ degenerated neurons were significantly more in Atf3 KO mice than that in WT mice after ischemic stroke, and that the injury areas were significantly larger and neurological deficits were more severe in Atf3 KO mice than WT control after SCI and ischemic stroke. Our study also shows that deletion of Atf3 gene significantly reduced the upregulation of SPRR1a, a molecule that promotes tissue regeneration (24), after CNS injuries, which likely contributes to the more severe neurological dysfunction and tissue damage in Atf3 KO mice after SCI and stroke. In other words, not to be bound by theory, it is likely that ATF3, a transcription factor induced in neurons after injury, mediates its neuroprotective function by stimulating the expression of regeneration-associated genes like Sprr1a.

### Example 2. Levels of ATF3 in Human SCI Patients, Stroke Patients, and Patient Having Cardiac Arrest

### AFT3 Levels in Human Serum—SCI Patients

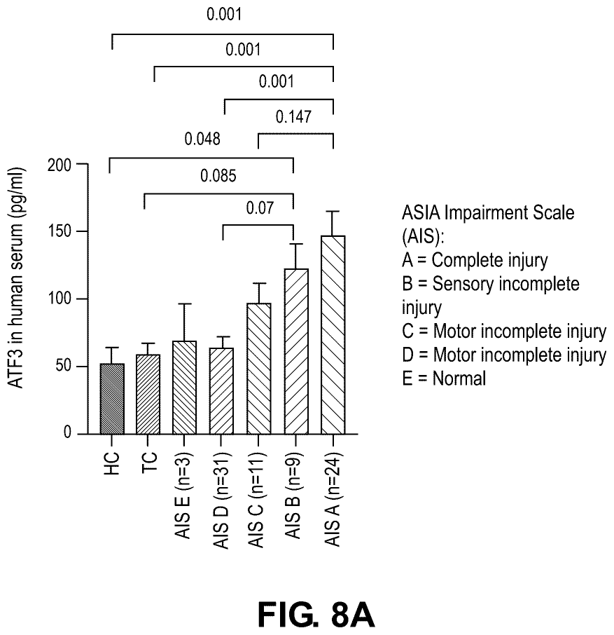

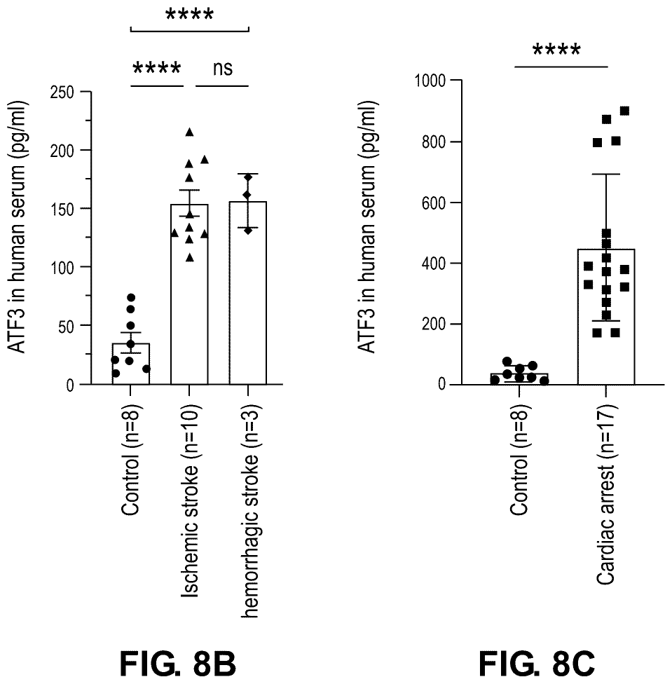

ATF3 levels in human serum were evaluated by ELISA using a commercially available kit in spinal cord injury patients 24 hours post-injury (FIG. 8A) compared to respective control patients. Injury severity in the patient was assigned using the Americal Spinal Injury Association (ASIA) Impairment Scale:
Grade A: The impairment is complete. No motor or sensory function below the level of injury.
Grade B: The impairment is incomplete. Sensory function, but not motor function, is preserved below the neurologic level (the first normal level above the level of injury) and some sensation is preserved in the sacral segments S4 and S5.
Grade C: The impairment is incomplete. Motor function is preserved below the neurologic level, but more than half of the key muscles below the neurologic level have a muscle grade less than 3 (i.e., they are not strong enough to move against gravity).
Grade D: The impairment is incomplete. Motor function is preserved below the neurologic level, and at least half of the key muscles below the neurologic level have a muscle grade of 3 or more (i.e., the joints can be moved against gravity).
Grade E: The patient's functions are normal.

The results demonstrated that ATF3 levels were elevated after SCI and were injury-severity-dependent (one-way ANOVA with Tukey's test).

### AFT3 Levels in Human Serum—Stroke Patients

Human serum ATF3 levels were measured by ELISA in samples from controls and patient cohorts with ischemic and hemorrhagic stroke (FIG. 8B). Samples were collected at 24 hrs+/−11.4 post-stroke for ischemic stroke (IS) patients; and 17.3 h+/−8.3 post stroke for hemorrhagic stroke (HS) patients. The results demonstrated that serum ATF3 levels were significantly elevated in both groups of stroke patients (Age: Ischemic stroke (IS): 76.3+/−11.2; Hemorrhagic stroke (HS): 75.7+/−9.8; NIH stroke scale: IS: 12.7+/−11.2; HS: 17.3+/−11.7). Control patients have similar pass medical history such as cardiovascular disease, diabetes mellitus, hyperlipidemia, without stroke. Data are presented as mean±S.E.M., n=4-10 in each group, one-way ANOVA with Tukey's multiple comparison tests, ****p<0.0001, ns=not significant. The NIH stroke scale is: 1-4=minor stroke, 5-15=moderate stroke, 15-20=moderate/severe stroke, 21-42=severe stroke.

FT3 Levels in Human Serum—Patients with Cardiac Arrest

Human serum ATF3 levels were measured by ELISA in samples from controls and a patient cohort with cardiac arrest (FIG. 8C). Samples were collected 24 hours post-cardiac arrest. Patient with cardiac arrest underwent cardiopulmonary resuscitation and neurological function were assessed post arrest (data not shown). Control patients have similar pass medical history such as cardiovascular disease, diabetes mellitus, hyperlipidemia, without cardiac arrest. Serum ATF3 levels were significantly elevated in patients with cardiac arrest. Data are presented as mean±S.E.M., n=8-17 in each group, Unpaired t test, ****p<0.0001.

### Materials and Methods

SCI patients and controls enrollment. The clinical procedures for SCI study were conducted with the approval of the Human Subjects Review Boards at the University of California, San Francisco, and the U.S. Department of Defense Human Research Protection Office. Our SCI patients, trauma controls and healthy cohort were part of the Brain and Spinal Injury Center (BASIC) Transforming Research and Clinical Knowledge (TRACK)-SCI project (7, 60) (https://clinicaltrials.gov/ct2/show/NCT04565366). Blood samples were collected from four SCI patients with AIS B (ASIA-American Spinal Injury Association Impairment Scale) scores at 24 hours after injury, four trauma control patients without SCI or TBI at 24 hours after injury, and four healthy controls. Patients who presented to the emergency department and were diagnosed with a traumatic SCI were enrolled with ISNCSCI examinations, blood draws, and follow up assessments. The trauma control patients were recruited from emergency department patients with traumatic but non-CNS injuries. The same basic demographic and biospecimen data were collected for these patients and from healthy control as for SCI patients for comparison purposes.

Stroke patients and controls enrollment—The clinical procedures for stroke study were conducted with the approval of the Human Subjects Review Boards at the University of California, San Francisco. Our ischemic stroke patients were enrolled, and blood samples were collected in the intensive care units (ICU). Biospecimen and demographic information for control cohort having similar medical history (cardiovascular diseases, hyperlipidemia, diabetes mellitus etc.) without stroke were also collected for comparison.

Animals: C57BL/6J (WT) mice were purchased from the Jackson Laboratory (Bar Harbor, ME, USA). Long Evans rats were purchased from Charles Rivers (Boston, MA). Atf3 knockout (KO) mice were provided by Dr. Tsonwin Hai (18), and raised in the animal facility of Zuckerberg San Francisco General Hospital, University of California, San Francisco (UCSF). Animals were housed with ad libitum access to food and water (maximum 5 per cage for mice and 2 for rats). All experiments were performed in accordance with National Institutes of Health guidelines and were approved by Institutional Animal Care and Use Committees at UCSF. Eight to ten-week old WT and Atf3 KO mice or rats at 11 weeks±3 days were randomly assigned to different experimental groups. For mice, both male and female mice were used and analyzed together. For rats, only female rats were included.

### Neurological Models:

Spinal cord injury: SCI was performed as previously described (19). All surgical procedures were performed aseptically. Rats or mice were administered with cefazolin 50 mg/kg before surgery and one day post-SCI. Animals were anesthetized with 2% isoflurane and buprenorphine (0.1 mg/kg) was given before surgery and next day after surgery. A dorsal midline skin incision was made, and connective tissue and muscle layers were dissected and a C5 laminectomy was performed. Unilateral (right) cervical contusion injury at 75 Kdyne (rats) and 30 Kdyne (mice) was produced with an Infinite Horizon (IH) impactor device. The surgical control group had all procedures without contusion. After injury, muscle layers were sutured and skin incision closed with wound clips. Animals were placed and monitored in incubator at 37° C. for the first day post-SCI. Animals were then checked twice daily for bladder function and wound healing following SCI for up to 7 days.

Permanent distal middle cerebral artery occlusion (pMCAO): The pMCAO was performed as described previously (50). Briefly, a 1.0 cm skin incision was made from the left orbit to the ear, followed by craniotomy (2 mm²) to expose the distal branches of middle cerebral artery (MCA). The MCA was then permanently occluded by electrical coagulation just proximal to the pyriform branch. The surface cerebral blood flow was monitored by a laser Doppler flow-meter (Vasamedics, Little Canada, MN, USA). Mice were excluded if the reduction of surface cerebral blood flow in the ischemic core is <15% of the baseline or massive bleeding occurred. In this study, 8 mice were excluded and replaced by additional mice. Mice were allowed to recover from anesthesia in warm and clean cages. All surgeries were performed under anesthesia with 2% isoflurane inhalation and aseptic conditions. Buprenorphine (analgesia, 0.1 mg/kg of body weight) was given at the beginning of and 6 hours after the surgery and as needed afterward. Rectal temperature was maintained at 37±±0.5° C. using a thermal blanket during surgery. Sham control mice were subjected to craniotomy without arterial occlusion and were subjected to the same amount and duration of anesthesia and the same amount of buprenorphine as mice subjected to pMCAO.

### Behavioral Tests:

For Spinal cord injury mice: We employed two behavioral tests on WT and Atf3 KO mice SCI model: paw placement (19) and foot drop in grid walk (20). 1). Paw placement: frequency of contralateral (left) forepaw placement is calculated and used as an indicator for functional recovery on ipsilateral (right) side. 2). Foot drop during grid walk: while mice walk across the grid, the number of times the mouse slips its ipsilateral forepaw off the grid is counted and the frequency of foot drop among all the steps is calculated. Both tests were performed before SCI (baseline), 2 d, 7 d and 14 d post-SCI.

For pMCAO mice: 1). Adhesive removal test was performed to assess potential somatosensory dysfunction (21). Briefly, a piece of adhesive tape (0.3×0.3 cm) was placed on one of the forepaws, and the time the mouse took to remove the tape was recorded. The maximum testing time was 120 seconds(s). Mice were trained twice daily for 4 days before pMCAO procedure to obtain an optimal level of performance. The adhesive removal times were recorded after 1 day before pMCAO (baseline), and 3 days after pMCAO. In general, stroke mice will take longer-time to remove the tape from the paw on the opposite side of the stroke lesion. Since the infarct in our model was on the left side of the brain, the adhesive removal times from the right paw were more relevant. 2). Corner test was performed to detect sensorimotor and postural asymmetries after ischemic stroke (22). Mice were placed between two 30×20 cm boards. Both sides of their vibrissae were stimulated as they approached the corner. The mice then moved up and turn to face the open end. For normal mice, the frequency of right and left turns would be equal. The stroke mice could not sense the stimulation on the stroke side, and hence, they made more turns to the ipsilateral side of stroke lesion (to the left in this study). Three different sets of 10 trials were conducted. Turning not incorporated in a rearing movement was excluded.

RNA Preparation and qRT-PCR

Total cellular RNA was prepared as previously described (51). RNA samples were purified from a 5 mm segment of mouse spinal cord centered on the impact site (or at C5 for uninjured tissue) using Trizol (Invitrogen, Carlsbad, CA) followed by RNeasy (Qiagen, Valencia, CA) binding, and quantified by a NanoDrop Lite (Thermo Scientific). We assessed mRNA expression of target genes in uninjured and injured spinal cord (4 h or 1 d; n=3-4/group per time point) by quantitative reverse transcription PCR (qRT-PCR) (51) using selected gene-specific primer pairs. cDNA was prepared from a total of 2 μg RNA by reverse transcription with SuperScript II and random primers as suggested by the manufacturer (Invitrogen). The PCR reactions were performed using 10 ng of cDNA, 50 nm of each primer, and SYBR Green master mix (Applied Biosystems) in 20 μl reactions. Levels of qRT-PCR product were measured using SYBR Green fluorescence collected on an Agilent Mx3005P Real-Time PCR system. Standard curves were generated for each gene using a control cDNA dilution series. Melting point analyses were performed for each reaction to confirm single amplified products.

### RNA Sequencing (RNA-Seq)

Cellular RNA prepared from WT sham and injured mice were quantitated and 50 ng of RNA were submitted to UCSF Functional Genomic Core facility for our RNA-Seq. Illumina HiSeq 4000 sequencer was used with total of 12 samples in one lane. We performed 50 bp single-ended sequencing at 30 million reads per sample. UCSF functional genomic core facility delivered the data upon library submission. The data were then trimmed and aligned against mouse genome. We performed differential expression analyses with multiple comparisons (Benjamini-Hochberg false discovery rate (16); BHFDR, Adjusted p<0.05) on our samples. 177 genes (160 upregulated and 17 downregulated) with more than 1.5-fold changes were identified after BHFDR adjusted p<0.05). These DEG were submitted to Metascape (metascape.org) for gene ontology (GO) analysis (52). Heatmap and volcano plot were also performed for data visualization. We will deposit our sequence data in the Short Read Archive at the National Library of Medicine/NIH to provide a valuable new resource for the SCI community.

### Tissue Processing, Histological and Immunohistochemical Staining

Spinal cord injury animals (rats or mice) were sacrificed under deep anesthesia (ketamine and xylazine) by transcardiac perfusion with 0.9% saline followed by 4% paraformaldehyde (PFA) (51). Segments of spinal cord centered on the injection sites or lesion were removed and postfixed overnight in 4% paraformaldehyde. Tissue was cryoprotected in 30% sucrose for 2 d, cut into 10 mm blocks. Tissue was embedded in OCT and sectioned 20 μm horizontally, dividing adjacent sections across six sets of slides. Antibody labeling was performed on fixed tissue sections from a full set of experimental conditions simultaneously using a high-throughput staining station (Sequenza; Thermo Scientific).

For immunohistochemical staining (51), rat spinal tissue sections were blocked and permeabilized for 1 h with 10% normal donkey serum and 0.3% Triton X-100 before antibody application. Sections were incubated overnight at room temperature with a solution containing mouse monoclonal antibody for ATF3 (1:300, Novus Biologicals, CO), NeuN (1:500, Millipore, MA), CD68 (1:50, AbD Serotec, NC), GFAP (1:500, Calbiochem, CA) and Olig2 (1:500) antibodies. After four washes with 2 ml of PBS, all slides were incubated for 1 hour at RT with the same freshly diluted fluorescent secondary antibody solution containing 1:500 Alexa 488 and 1:500 Alexa 594 conjugated IgG (Invitrogen, CA). Slides were briefly rinsed with 2 ml PBS and coverslipped with Vectashield containing DAPI (4′,6-diamidino-2-phenylindole) (Vector Laboratories). Each immunolabel had three negative controls: no primary, and each individual primary with the incorrect secondary. Microscopy on these controls revealed no detectable label above threshold. The stained spinal tissue sections were photographed with a ×10 or ×40 objective using the BIOREVO all-in-one fluorescence microscope (BZ-9000 Generation II, Keyence microscope). The images of a cross section were stitched together, and positive signal was measured using BZ-9000 Generation II analyzer (Keynence).

pMCAO mice were anesthetized and brain tissues were collected and frozen on dry ice. A series of 20-μm-thick coronal sections were made between bregma −1.22 and −2.18 mm using a Leica Cryostat (CM1900, Wetzlar, Germany). After air dry and washed with phosphate-buffered saline (PBS), sections were stained with antibodies specific to CD68 (for activated microglia/macrophage, 1:50, AbD Serotec, MCA1957, Raleigh, NC, USA), NeuN (for neuronal nuclei, 1:500, Millipore, Bedford, MA), ATF3 (1:300, Novus Biologicals, Centennial, CO, USA) and Glial fibrillary acidic protein (GFAP, for astrocyte, 1:500, Calbiochem, La Jolla, CA, USA) at 4° C. overnight and then with secondary antibody, Alexa Fluor 594-conjugated IgG or Alexa Fluor 488-conjugated IgG (1:500, Invitrogen, Carlsbad, CA, USA) at room temperature for 2 hours. Fluoro-Jade C (Millipore, Bedford, MA, USA) staining was performed according to the manufacturer instruction. Sections were mounted with Vectashield HardSet Mounting Medium with Dapi (Vector Laboratories Inc, Burlingame, CA, USA).

### Injury Size Estimation:

The lesion volume of spinal cord injured mice was assessed on paraformaldehyde (PFA)-fixed tissue collected at the end of 2 weeks following mouse SCI (51). Eriochrome cyanine (EC) staining was used to differentiate spinal tissues (23). A camera lucida drawing of the section with the largest lesion (epicenter) is made outlining intact gray and white matter, and the lesion. Tissue areas in which normal spinal cord architecture was absent and/or demyelination or fibrosis was present were defined as lesion. These areas were outlined manually from digitized images. The percentage of lesion volume was calculated accordingly.

The infarct volumes were measured on pMCAO mice 3 days after the injury. One of every 10 brain sections (200 μm apart) was selected, stained with cresyl violet and imaged. The infarct areas were outlined and quantified using IMAGE J (National Institutes of Health, Bethesda, MD, USA). The infarct volumes were calculated by multiplying the sum of infarct areas from all cresyl violet-stained sections by 200.

CSF and blood collection: At different time points, sham and injured mice were anesthetized and cisterna magna was surgically exposed. Capillary tube was inserted through dura gently without damaging adjacent blood vessels. Typically, 2-5 ul CSF can be collected, which were quickly frozen on dry ice and stored in −80° C. After CSF collection, the same animal was deep anesthetized with ketamine and xylazine and peripheral blood was collected via transcardiac puncture. Typically, 200-500 μl blood can be obtained. After blood collection, blood samples were centrifuged 3000 rpm/min for 15 min and upper layer containing plasma was removed and stored at −80° C.

ATF3 ELISA: Commercial mouse, rat (MyBioSource Inc., CA) and human ATF3 (Aviva Systems Biology, CA) ELISA kits were used to quantitate ATF3 protein levels in spinal tissue, CSF and plasma (rodent SCI) or serum (human samples) based on standard sandwich enzyme-linked immune-sorbent assay technology. For human serum samples, they were prepared by separating clot using serum separator tube (SST) for 15 minutes and brief centrifuge and tested by human ATF3 ELISA kit (Cat #OKDD01469, Aviva Systems Biology Corp. San Diego, CA).

Standards or test samples were assayed following the manufacturer's procedures. Typically, all wells in the micro-well plate were precoated with anti-human ATF3 antibody. Samples for standard curve were reconstituted from standard stock solution as 1000 pg/mL, 500 pg/mL, 250 pg/mL, 125 pg/mL, 62.5 pg/mL, 31.2 pg/mL, 15.6 pg/mL, and blank (0 pg/mL), and the testing spinal cord, CSF, plasma, or serum samples were used with or without dilution, with duplicates for each standard and testing samples. The micro-well plate was covered with plate sealer before each incubation at 37° C. After adding the standard and testing samples into the precoated wells, the plate was incubated at 37° C. for 90 minutes. 100 μL of biotinylated detection antibody was then added to each well after the liquid was removed, and the samples were incubated at 37° C. for 45 minutes. After the aspiration/wash process for a total of 3 times, 100 μL of Avidin-HRP conjugate was added to each well, and the plate was incubated at 37° C. for 45 minutes. After another aspiration/wash process for 5 times, 90 μL of substrate solution was added to each well, with protection from light, and the plate was incubated at 37° C. for 15-25 minutes. 50 μL of stop solution was then added to each well, and the samples were measures at 450 nm through the microplate reader.

### Statistical Analyses:

Spinal cord injury: Two-way ANOVA with repeated measures was used for behavioral and histological analysis. Terminal histology, qRT-PCR, western blot and ELISA results were analyzed by either two-way ANOVA with post hoc test for three groups or unpaired two-tailed t-tests for two group analyses. GraphPad Prism 8 was used for graphs and data analyses (GraphPad Software, La Jolla, CA). Data are expressed as means±s.e.m. Statistical significance was defined at p≤0.05, 0.01, 0.001 or 0.0001 levels. Statistical results are presented in the figure legends.

pMCAO: Data are presented as mean±s.e.m. Sample size were estimated according to our previous published effect sizes of infarct volume and sensorimotor function in a similar model (53, 54) and were indicated in the figure legends. All quantification analyses were performed by at least two researchers who did not know the group assignment. Two group comparisons were analyzed using unpaired t-test using GraphPad Prism 6. Multiple group comparisons were analyzed by one-way ANOVA with Tukey's multiple comparisons, except behavior tests, which were analyzed by Two-way ANOVA with Tukey's multiple comparisons. p value≤0.05 was considered to be statistically significant.

### REFERENCES CITED BY CITATION NUMBER IN SPECIFICATION

1. Apple F S. Clinical biomarkers of cardiac injury: cardiac troponins and natriuretic peptides. Toxicol Pathol. 2006; 34 (1): 91-3.
2. German C A, and Shapiro M D. Assessing Atherosclerotic Cardiovascular Disease Risk with Advanced Lipid Testing: State of the Science. Eur Cardiol. 2020; 15: e56.
3. Grunnet M, and Sorensen J B. Carcinoembryonic antigen (CEA) as tumor marker in lung cancer. Lung Cancer. 2012; 76 (2): 138-43.
4. Hayes J H, and Barry M J. Screening for prostate cancer with the prostate-specific antigen test: a review of current evidence. JAMA. 2014; 311 (11): 1143-9.
5. Laterza O F, Lim L, Garrett-Engele P W, Vlasakova K, Muniappa N, Tanaka W K, et al. Plasma MicroRNAs as sensitive and specific biomarkers of tissue injury. Clin Chem. 2009; 55 (11): 1977-83.
6. Rodrigues L F, Moura-Neto V, and TCLS ES. Biomarkers in Spinal Cord Injury: from Prognosis to Treatment. Mol Neurobiol. 2018; 55 (8): 6436-48.
7. Kyritsis N, Torres-Espin A, Schupp P G, Huie J R, Chou A, Duong-Fernandez X, et al. Diagnostic blood RNA profiles for human acute spinal cord injury. J Exp Med. 2021; 218 (3).
8. Wang K K, Yang Z, Zhu T, Shi Y, Rubenstein R, Tyndall J A, et al. An update on diagnostic and prognostic biomarkers for traumatic brain injury. Expert Rev Mol Diagn. 2018; 18 (2): 165-80.
9. Dagonnier M, Donnan G A, Davis S M, Dewey H M, and Howells D W. Acute Stroke Biomarkers: Are We There Yet? Front Neurol. 2021; 12:619721.
10. Kwon B K, Bloom O, Wanner I B, Curt A, Schwab J M, Fawcett J, et al. Neurochemical biomarkers in spinal cord injury. Spinal Cord. 2019; 57 (10): 819-31.
11. Yokobori S, Zhang Z, Moghieb A, Mondello S, Gajavelli S, Dietrich W D, et al. Acute diagnostic biomarkers for spinal cord injury: review of the literature and preliminary research report. World Neurosurg. 2015; 83 (5): 867-78.
12. Bazarian J J, Biberthaler P, Welch R D, Lewis L M, Barzo P, Bogner-Flatz V, et al. Serum GFAP and UCH-L1 for prediction of absence of intracranial injuries on head CT (ALERT-TBI): a multicentre observational study. Lancet Neurol. 2018; 17 (9): 782-9.
13. Li Y, Chen Y, Li X, Wu J, Pan J Y, Cai R X, et al. RNA sequencing screening of differentially expressed genes after spinal cord injury. Neural Regen Res. 2019; 14 (9): 1583-93.
14. Chen K, Deng S, Lu H, Zheng Y, Yang G, Kim D, et al. RNA-seq characterization of spinal cord injury transcriptome in acute/subacute phases: a resource for understanding the pathology at the systems level. PloS one. 2013; 8 (8): e72567.
15. Shi L L, Zhang N, Xie X M, Chen Y J, Wang R, Shen L, et al. Transcriptome profile of rat genes in injured spinal cord at different stages by RNA-sequencing. BMC Genomics. 2017; 18 (1): 173.
16. Hochberg Y, and Benjamini Y. More powerful procedures for multiple significance testing. Stat Med. 1990; 9 (7): 811-8.
17. Schmued L C, Stowers C C, Scallet A C, and Xu L. Fluoro-Jade C results in ultra high resolution and contrast labeling of degenerating neurons. Brain Res. 2005; 1035 (1): 24-31.
18. Hartman M G, Lu D, Kim M L, Kociba G J, Shukri T, Buteau J, et al. Role for activating transcription factor 3 in stress-induced beta-cell apoptosis. Mol Cell Biol. 2004; 24 (13): 5721-32.
19. Gensel J C, Tovar C A, Hamers F P, Deibert R J, Beattie M S, and Bresnahan J C. Behavioral and histological characterization of unilateral cervical spinal cord contusion injury in rats. Journal of neurotrauma. 2006; 23 (1): 36-54.
20. Luong T N, Carlisle H J, Southwell A, and Patterson P H. Assessment of motor balance and coordination in mice using the balance beam. Journal of visualized experiments: JoVE. 2011 (49).
21. Bouet V, Freret T, Toutain J, Divoux D, Boulouard M, and Schumann-Bard P. Sensorimotor and cognitive deficits after transient middle cerebral artery occlusion in the mouse. Experimental neurology. 2007; 203 (2): 555-67.
22. Zhang L, Schallert T, Zhang Z G, Jiang Q, Arniego P, Li Q, et al. A test for detecting long-term sensorimotor dysfunction in the mouse after focal cerebral ischemia. J Neurosci Methods. 2002; 117 (2): 207-14.
23. Irvine K A, Ferguson A R, Mitchell K D, Beattie S B, Lin A, Stuck E D, et al. The Irvine, Beatties, and Bresnahan (IBB) Forelimb Recovery Scale: An Assessment of Reliability and Validity. Front Neurol. 2014; 5:116.
24. Bonilla I E, Tanabe K, and Strittmatter S M. Small proline-rich repeat protein 1A is expressed by axotomized neurons and promotes axonal outgrowth. The Journal of neuroscience: the official journal of the Society for Neuroscience. 2002; 22 (4): 1303-15.
25. Hunt D, Raivich G, and Anderson P N. Activating transcription factor 3 and the nervous system. Frontiers in molecular neuroscience. 2012; 5:7.
26. Courtine G, and Sofroniew M V. Spinal cord repair: advances in biology and technology. Nature medicine. 2019.
27. Venkatesh K, Ghosh S K, Mullick M, Manivasagam G, and Sen D. Spinal cord injury: pathophysiology, treatment strategies, associated challenges, and future implications. Cell Tissue Res. 2019.
28. Bustamante A, Garcia-Berrocoso T, Rodriguez N, Llombart V, Ribo M, Molina C, et al. Ischemic stroke outcome: A review of the influence of post-stroke complications within the different scenarios of stroke care. Eur J Intern Med. 2016; 29:9-21.
29. Feigin V L, Lawes C M, Bennett D A, Barker-Collo S L, and Parag V. Worldwide stroke incidence and early case fatality reported in 56 population-based studies: a systematic review. Lancet Neurol. 2009; 8 (4): 355-69.
30. Fricker M, Tolkovsky A M, Borutaite V, Coleman M, and Brown G C. Neuronal Cell Death. Physiol Rev. 2018; 98 (2): 813-80.
31. Stoica B A, and Faden A I. Cell death mechanisms and modulation in traumatic brain injury. Neurotherapeutics. 2010; 7 (1): 3-12.
32. Bredesen D E. Programmed cell death mechanisms in neurological disease. Curr Mol Med. 2008; 8 (3): 173-86.
33. Bredesen D E. Key note lecture: toward a mechanistic taxonomy for cell death programs. Stroke. 2007; 38 (2 Suppl): 652-60.
34. Beattie M S, Hermann G E, Rogers R C, and Bresnahan J C. Cell death in models of spinal cord injury. Progress in brain research. 2002; 137:37-47.
35. Gutierrez I L, Gonzalez-Prieto M, Garcia-Bueno B, Caso J R, Leza J C, and Madrigal J L M. Alternative Method to Detect Neuronal Degeneration and Amyloid beta Accumulation in Free-Floating Brain Sections With Fluoro-Jade. ASN Neuro. 2018; 10:1759091418784357.
36. Schmued L C, Albertson C, and Slikker W, Jr. Fluoro-Jade: a novel fluorochrome for the sensitive and reliable histochemical localization of neuronal degeneration. Brain Res. 1997; 751 (1): 37-46.
37. Ikenari T, Kurata H, Satoh T, Hata Y, and Mori T. Evaluation of Fluoro-Jade C Staining: Specificity and Application to Damaged Immature Neuronal Cells in the Normal and Injured Mouse Brain. Neuroscience. 2020; 425:146-56.
38. Kumar S. Caspase function in programmed cell death. Cell Death Differ. 2007; 14 (1): 32-43.
39. Perng G C, Jones C, Ciacci-Zanella J, Stone M, Henderson G, Yukht A, et al. Virus-induced neuronal apoptosis blocked by the herpes simplex virus latency-associated transcript. Science. 2000; 287 (5457): 1500-3.
40. Hai T, Wolfgang C D, Marsee D K, Allen A E, and Sivaprasad U. ATF3 and stress responses. Gene Expr. 1999; 7 (4-6): 321-35.
41. Tsujino H, Kondo E, Fukuoka T, Dai Y, Tokunaga A, Miki K, et al. Activating transcription factor 3 (ATF3) induction by axotomy in sensory and motoneurons: A novel neuronal marker of nerve injury. Mol Cell Neurosci. 2000; 15 (2): 170-82.
42. Renthal W, Tochitsky I, Yang L, Cheng Y C, Li E, Kawaguchi R, et al. Transcriptional Reprogramming of Distinct Peripheral Sensory Neuron Subtypes after Axonal Injury. Neuron. 2020; 108 (1): 128-44 e9.
43. Seijffers R, Allchorne A J, and Woolf C J. The transcription factor ATF-3 promotes neurite outgrowth. Mol Cell Neurosci. 2006; 32 (1-2): 143-54.
44. Seijffers R, Mills C D, and Woolf C J. ATF3 increases the intrinsic growth state of DRG neurons to enhance peripheral nerve regeneration. The Journal of neuroscience: the official journal of the Society for Neuroscience. 2007; 27 (30): 7911-20.
45. Wang L F, Huang S B, Zhao H D, Liu C J, Yao L, and Shen Y Q. Activating transcription factor 3 promotes spinal cord regeneration of adult zebrafish. Biochem Biophys Res Commun. 2017; 488 (3): 522-7.
46. Forstner P, Rehman R, Anastasiadou S, Haffner-Luntzer M, Sinske D, Ignatius A, et al. 46. Neuroinflammation after Traumatic Brain Injury Is Enhanced in Activating Transcription Factor 3 Mutant Mice. Journal of neurotrauma. 2018; 35 (19): 2317-29.
47. Takarada T, Kou M, Hida M, Fukumori R, Nakamura S, Kutsukake T, et al. Protective upregulation of activating transcription factor-3 against glutamate neurotoxicity in neuronal cells under ischemia. Journal of neuroscience research. 2016; 94 (5): 378-88.
48. Wang L, Deng S, Lu Y, Zhang Y, Yang L, Guan Y, et al. Increased inflammation and brain injury after transient focal cerebral ischemia in activating transcription factor 3 knockout mice. Neuroscience. 2012; 220:100-8.
49. Seijffers R, Zhang J, Matthews J C, Chen A, Tamrazian E, Babaniyi O, et al. ATF3 expression improves motor function in the ALS mouse model by promoting motor neuron survival and retaining muscle innervation. Proceedings of the National Academy of Sciences of the United States of America. 2014; 111 (4): 1622-7.
50. Wang L, Kang S, Zou D, Zhan L, Li Z, Zhu W, et al. Bone Fracture Pre-Ischemic Stroke Exacerbates Ischemic Cerebral Injury in Mice. PloS one. 2016; 11 (4): e0153835.
51. Gao J, Sun Z, Xiao Z, Du Q, Niu X, Wang G, et al. Dexmedetomidine modulates neuroinflammation and improves outcome via alpha2-adrenergic receptor signaling after rat spinal cord injury. Br J Anaesth. 2019; 123 (6): 827-38.
52. Zhou Y, Zhou B, Pache L, Chang M, Khodabakhshi A H, Tanaseichuk O, et al. Metascape provides a biologist-oriented resource for the analysis of systems-level datasets. Nat Commun. 2019; 10 (1): 1523.
53. Shen F, Jiang L, Han F, Degos V, Chen S, and Su H. Increased Inflammatory Response in Old Mice is Associated with More Severe Neuronal Injury at the Acute Stage of Ischemic Stroke. Aging Dis. 2019; 10 (1): 12-22.
54. Shen F, Degos V, Chu P L, Han Z, Westbroek E M, Choi E J, et al. Endoglin deficiency impairs stroke recovery. Stroke. 2014; 45 (7): 2101-6.

It is understood that the examples and embodiments described herein are for illustrative purposes only and that various modifications or changes in light thereof will be suggested to persons skilled in the art and are to be included within the spirit and purview of this application and scope of the appended claims. All publications, patents, accession numbers, and patent applications cited herein are hereby incorporated by reference in their entirety for all purposes.

1. A method of assessing severity of a spinal cord injury (CSI), the method comprising
determining the level of ATF3 polypeptide in a cerebrospinal fluid (CSF) or blood sample from a subject that has a CSI;
comparing the level of ATF3 polypeptide to a reference scale determined from a population of patients having American Spinal Injury Association Impairment Scale (AIS) Grades ranging from Grade A to Grade E; and
classifying the severity of the CSI, wherein:
the subject is classified as having severe injury if the level of ATF3 polypeptide exceeds a threshold value for Grade A injury;
the subject is classified as having moderate injury if the level of ATF3 polypeptide exceeds a threshold value for Grade C injury, but is less than the threshold value for Grade A injury; or
the subject is classified as having mild injury if the level of ATF3 polypeptide is below the threshold value for Grade C injury.

2. The method of claim 1, wherein the sample is a CSF sample.

3. The method of claim 1, wherein the sample is a blood sample.

4. A method of assessing severity of a central nervous system injury, the method comprising
determining the level of ATF3 polypeptide or RNA in a cerebrospinal fluid (CSF) or blood sample from a subject that has a CNS injury;
comparing the level of ATF3 polypeptide or RNA to a reference scale derived from a population of patients having mild to severe CNS injury; and
classifying the severity of injury, wherein:
the subject is classified as having severe injury if the level of ATF3 polypeptide or RNA is in the top tertile of the reference scale;
the subject is classified as having moderate injury if the level of ATF3 polypeptide or RNA is in the middle tertile of the reference scale; or
the subject classified as having mild injury if the level of ATF3 polypeptide or RNA is in the lowest tertile of the reference scale.

5. A method of assessing severity of a central nervous system injury, the method comprising
determining the level of ATF3 polypeptide or RNA in a cerebrospinal fluid (CSF) or blood sample from a subject that has a CNS injury;
comparing the level of ATF3 polypeptide or RNA to a reference scale derived from a population of patients having mild to severe CNS injury; and
classifying the severity of injury, wherein:
the subject is classified as having severe injury if the level of ATF3 falls within one standard deviation of a value on the reference scale associated with severe injury;
the subject is classified as having moderate injury if the level of ATF3 falls within one standard deviation of a value on the reference scale associated with moderate injury; or
the subject is classified as having mild injury if the level of ATF3 falls within one standard deviation of a value on the reference scale associated with mild injury.

6. The method of claim 4, wherein the CNS injury is a spinal cord injury (CSI).

7. The method of claim 4, wherein the sample is a blood sample.

8. The method of claim 4, wherein the sample is a CSF sample.

9. The method of claim 4, wherein the method comprises determining the level of ATF3 polypeptide.

10. The method of claim 4, wherein the CNS injury is a stroke.

11. The method of claim 10, wherein the stroke is an ischemic stroke.

12. The method of claim 10, wherein the stroke is a hemorrhagic stroke.

13. The method of claim 10, wherein the sample is a blood sample.

14. The method of claim 4, wherein the patient has suffered from cardiac arrest.

15. The method of claim 10, wherein the method comprises determining the level of ATF3 polypeptide.

16. A method of detecting ATF3 in a CSF sample or blood sample from a subject having a CNS injury, the method comprising contacting the CSF sample or blood sample with an agent that specifically binds to ATF3; and detecting the agent bound to ATF3.

17. The method of claim 16, wherein the sample is a CSF sample.

18. The method of claim 16, wherein the CNS injury is a spinal cord injury.

19. The method of claim 16, wherein the CNS injury is a stroke.

20. The method of claim 19, wherein the stroke is an ischemic stroke.

21. The method of claim 19, wherein the stroke is a hemorrhagic stroke.

22. The method of claim 14, wherein the patient had cardiac arrest.
